(19) United States
(12) Patent Application Publication
(10) Pub. No.: US 2024/0058630 A1
(43) Pub. Date: Feb. 22, 2024

(54) Personal Protection Air-Shield Devices and Uses Thereof

(71) Applicant: Hellene Coroneo, Randwick, New South Wales (AU)

(72) Inventor: Hellene Coroneo, Randwick, New South Wales (AU)

(21) Appl. No.: 18/267,698

(22) PCT Filed: Dec. 21, 2021
(86) PCT No.: PCT/IB2021/062093

§ 371 Date: Jun. 15, 2023

Related U.S. Application Data
(60) Provisional application No. 63/129,102, filed on Dec. 22, 2020.

(51) Int. Cl. A62B 18/00; A42B 1/0181; A41D 13/11

(52) U.S. Cl. CPC A62B 18/003; A42B 1/0181; A41D 13/1184

(57) ABSTRACT

The present invention relates to the field of mitigating or preventing infection. In particular, provided is a personal protection air-shield device capable of emitting laminar air, such as clean, ionized laminar air, and methods of using the same.

### CROSS REFERENCE TO RELATED APPLICATIONS

This application claims the benefit of priority under 35 U.S.C. § 119(e) from U.S. Provisional Application No. 63/129,102, filed on Dec. 22, 2020. The foregoing related application, in its entirety, is incorporated herein by reference.

### FIELD

The present invention relates to the field of mitigating or preventing infection. In particular, provided is a personal protection air-shield device capable of emitting laminar air, such as clean, ionized laminar air, and methods of using the same.

### SUMMARY

Provided herein is an air-shield device comprising: i) an airflow-generating device; and ii) a laminar airflow device having a vent or a plurality of exit ports, wherein the laminar airflow device is in communication with the airflow-generating device; wherein the air-shield device is a personal protection air-shield device.

Provided herein are methods of using the air-shield device disclosed herein. Provided herein is a method of protecting the eye-zone or T-zone of a subject in need, comprising the subject wearing the personal protection air-shield device disclosed herein. Provided herein is a method of protecting eyes, nose, or mouth of a subject in need, comprising the subject wearing the personal protection air-shield device disclosed herein.

Provided herein is a method of mitigating or preventing infection via the eye-zone or T-zone of a subject in need, comprising the subject wearing the personal protection air-shield device disclosed herein. Provided herein is a method of mitigating or preventing infection of eyes, nose, or mouth of a subject in need, comprising the subject wearing the personal protection air-shield device disclosed herein.

Provided herein is a method of protecting the eye-zone or T-zone of a subject in need, comprising emitting a laminar airflow that deflects or prevents surrounding external air or air currents from contacting the eye-zone or the T-zone of the subject. Provided herein is a method of protecting eyes, nose, or mouth of a subject in need, comprising emitting a laminar airflow that deflects or prevents surrounding external air or air currents from contacting the eyes, nose, or mouth of the subject.

### BRIEF DESCRIPTION OF THE DRAWINGS

FIG. 1. A graphic illustrating the T-zone, the region of the eyes, nose and mouth, portals for viral invasion.

FIGS. 2A-2B. FIG. 2A: Shows the top side of two styles of head-coverings (hats) fitted with a laminar airflow device (tubing attached to the underside of the rim). The tubing is perforated to allow flow of air vertically down and also angled away from the wearer. FIG. 2B: Shows the underside of the two styles of head-coverings (hats) fitted with a laminar airflow device (tubing attached to the underside of the rim). The tubing is perforated to allow flow of air vertically down and also angled away from the wearer.

FIGS. 3A-3B. FIG. 3A: Shows a laminar airflow device (tubing) connected to a airflow-generating device (rechargeable fan) attached to the underside of the rim of a head-covering (hat). FIG. 3B: Shows perforations in the laminar airflow device (tubing).

FIGS. 4A-4B. FIG. 4A: Shows a profile and slight underside view of an air-shield device having airflow-generating devices (fans) in series connected to the laminar airflow device (tubing) on the rim of the head-covering (hat) and connected to a rechargeable battery. FIG. 4B: Shows the underside view of the air-shield device having airflow-generating devices (fans) in series connected to the laminar airflow device (tubing) on the rim of the head-covering (hat) and connected to a rechargeable battery.

FIG. 5. A graphic illustrating a head-covering (hat) within a head-covering (hat) arrangement to allow emission of laminated air (e.g., a halo of laminated air) through 360 degrees.

FIGS. 6A-6B. FIG. 6A: Shows smoke striking the face of a mannequin wearing an air-shield device when the airflow-generating device (fan) is turned off. FIG. 6B: Shows smoke not reaching the mannequin (e.g., the face of the mannequin) wearing an air-shield device when the airflow-generating device (fan) is turned on.

FIGS. 7A-7B. FIG. 7A: A graphic illustrating air currents and routes taken by the naturally convected air over the body in a standing (right) or sitting (left) position. FIG. 7B: A graphic illustrating air currents and routes taken by the naturally convected air over the head.

FIGS. 8A-8C. FIG. 8A: A graphic illustrating human convective boundary; a profile of a face in a schlieren image during expiration. The white layer around the head is the inner part of the boundary layer, showing thickening of this layer in the region of the eyes. FIG. 8B: A graphic illustrating redirection of expired air by mask. FIG. 8C: A graphic illustrating the effect of downward air flow on the human facial convective air flow. Downward airflow is ˜3.5 cm from the eye, thereby avoiding dryness and upward flow form the nose, which may be beneficial to the ocular surface. Air from the peripheral rim of the device is aimed at the area of the chin, to deflect the normal (potentially viral laden air) from the lower part of the body.

FIGS. 9A-9B: Shows a front view (FIG. 9A) and a side view (FIG. 9B) of a spectacle device (a light spectacle frame) silhouetted with a perforated laminar airflow device (tubing).

FIGS. 10A-10B. FIG. 10A: Shows a front view (FIG. 10A) and under view (FIG. 10B) of failure of conventional side-wrap spectacles to block air access, particularly from below and from the sides.

FIGS. 11A-11B. FIG. 11A: Shows smoke striking the container fitted with wearing an air-shield device comprising a spectacle device when the airflow-generating device (fan) is turned off. FIG. 11B: Shows smoke not reaching the container fitted with wearing an air-shield device when the airflow-generating device (fan) is turned on, wherein the exclusion of the smoke at the inferotemproal aspect of the spectacle device by laminar air emitted from this aspect of the air-shield device.

FIG. 12. Shows the “internal” headband to separate head from a head-covering (hat).

FIG. 13. Shows a laminar airflow device comprising two sets of tubing perforated to allow flow of air in one or more directions, such as a combination of vertically downward, angled away from the wearer, and or angled towards the wearer.

FIG. 14. Shows the side view of a head-covering (hat) fitted with an air filtration system, said air filtration system comprising chambers housing air filters that filter and allow airflow to a laminar airflow device (tubing attached to the underside of the rim).

FIG. 15. Shows bottom view of a head-covering (hat) fitted with a laminar airflow device (tubing attached to the underside of the rim) comprising a UVC device (LEDs), wherein the sidewalls forming the tubing of the laminar airflow device are opaque to UVC light.

### DETAILED DESCRIPTION

As used herein, the articles “a,” “an,” and “the” refer to one or to more than one of the grammatical object of the article. By way of example, a sample refers to one sample or two or more samples.

As used herein, the term “subject” refers to a mammal. A subject can be a human or a non-human mammal such as a dog, cat, bovid, equine, mouse, rat, rabbit, or transgenic species thereof. The subject may have been exposed to a microbe, a bacterium, a virus, such as a coronavirus, for example, COVID-19 virus, or such as an adenovirus or a rhinovirus, for example, influenza virus. In some embodiments, the subject may have a bacterial infection or a virus infection, such as a coronavirus infection, for example, COVID-19 infection, or such as a rhinovirus infection, for example, influenza infection. The subject can be a patient who has a COVID-19 infection (e.g., has been diagnosed with a COVID-19 infection), or the subject can be a patient who has been exposed to a COVID-19 virus, but does not show one or more symptoms of having a COVID-19 infection.

COVID-19 aerosol and fomite transmission may be high, since this coronavirus is reportedly able to remain viable and infectious in aerosols for hours and on surfaces for up to days. Other viruses, such as rhinoviruses, for example, the infectious influenza virus (both as fine- and coarse-aerosol viral RNA), have been found in the exhaled breath of symptomatic seasonal influenza patients. According to this study, sneezing was rare, and sneezing and coughing were not necessary for infectious aerosol generation.

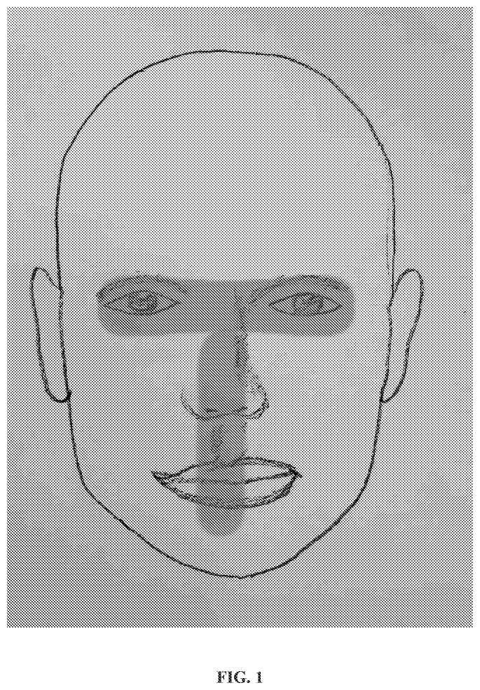

An unexpected consequence of the COVID-19 pandemic has been to highlight the inadequacy of personal protection from microbial and in particular viral invasion. Rapid and almost universal transmission of a highly contagious and malicious virus that can remain in the air for periods of up to 16 hours (Fears, A. C, et al., medRxiv [Preprint]. 2020 Apr. 18:2020.04.13.20063784) and can remain viable isolated for up to 28 days on surfaces such as glass, stainless steel and paper and polymer banknotes (Riddell, S., et al., Virol J, 2020; 17:145.) has resulted in a scramble for self-preservation and a shortage of personal protective equipment (PPE). Whereas in the past failure of this protection might result in a dose of influenza, now it could be a life-threatening experience. There is a desperate need for an effective, efficient, affordable, modern form of PPE that will eliminate the need for social isolation in crowded environments such as inflight and in other crowded spaces with relatively poor ventilation. Many of the protective devices in use were not designed for the task to which they currently have been put and their efficacy has been questioned. A “brute-force” approach involving physical barriers, “masking” the presumed portals of viral entry (nose, mouth and eyes), now termed the T-zone (or Ti-zone, Spencer, S. K. R., et al., Submitted to The Ocular Surface in October 2020; unpublished)—FIG. 1, remains in use and does not seem to have changed significantly from the attire of the plague doctors of C17th (Coroneo, M. T., Ocul Surf., 2020:S1542 0124(20)30089-6., Oldfield, E., et al., ACS Infect Dis., 2020; 6:1563-1566). The likely false presumption that viral entry is predominantly via the mouth and nose (Coroneo, M. T., Ocul Surf., 2020:S1542-0124(20)30089-6), has resulted in the use of face masks of variable protective capacity, the chronic use of which can result in inadequate protection and misuse.

Supposedly, masks serve a dual purpose of both preventing the spread of aerosolized droplets by the wearer and preventing inhalation of potentially infectious droplets from others. However, a recent meta-analysis concluded that the wearing of surgical masks by individuals in non-healthcare settings is not significantly associated with reduction in acute respiratory illness incidence (Wang, M. X., et al., Front Med., 2020; 7:564280).

Furthermore, a study of mask efficacy during influenza seasons of 2011-2015 found that there was no significant difference in the incidence of laboratory-confirmed influenza among two groups in outpatient health care personnel (8.2% for the N95 respirator and 7.2% for masks). When asked whether they wore the device “always” or “sometimes,” 89.4% of the respirator group did and 90.2% of the mask group did. Reasons for poor compliance when using respirators were that they were uncomfortable and hot, the inside rapidly became wet, it was nearly impossible to speak with them on, and when properly fitted it was extremely difficult to breathe (Radonovich, L. J. Jr., et al., JAMA., 2019; 322:824-833., Radonovich, L. J., et al., PLoS ONE., 2019; 14(1):e0209559.). Also, the wearing goggles, a face shield, or even eyeglasses might pose an increased risk of touching one's eyes more frequently and potentially contaminating them when removing, replacing, or adjusting the eye protection, especially if a person is not accustomed to wearing them. Self-contamination when doffing PPE is a well-documented risk that must be carefully considered before advising people to wear a new type of PPE (Maragakis, L L., JAMA Ophthalmol., 2019; Published online Sep. 16, 2020., Andonian, J., et al., Clin Infect Dis., 2019; 69(suppl 3):5248-5255.).

Prolonged use of masks can also result in causation/exacerbation of skin conditions (Hadjieconomou, S., et al., J Eur Acad Dermatol Venereol., 2020 Jun. 14:10.1111/jdv.16754. doi: 10.1111/jdv.16754. Epub ahead of print.) and may be difficult to use in those with respiratory illness (Institute of Medicine (US) Committee on Personal Protective Equipment for Healthcare Personnel to Prevent Transmission of Pandemic Influenza and Other Viral Respiratory Infections: Current Research Issues; Larson E L, Liverman C T, editors. Preventing Transmission of Pandemic Influenza and Other Viral Respiratory Diseases: Personal Protective Equipment for Healthcare Personnel: Update 2010. Washington (DC): National Academies Press (US); 2011.). Furthermore, outer surface viral contamination of masks used by hospital healthcare workers has been documented and may result in self-contamination. The risk is higher with longer duration of mask use (>6 h) and with higher rates of clinical contact (Chughtai, A. A., et al., BMC Infect Dis., 2019; 19:491.). This is problematic given that spontaneous face-touching is an ingrained habit for humans, who engage in this activity on average 17-23 times per hour (Morita K., et al., E3S Web of Conferences., 2019; 111:02027. Available from: http://dx.doi.org/10.1051/e3 sconf/201911102027., Kwok, Y. L. A., Am J Infect Control., 2015; 43:112-4.) and that nearly half of spontaneous face-touches involve mucous membranes, and one third of those involve the eyes. One potential benefit of mask wearing is that it significantly decreases the rate of face-touching in health professionals (Spencer, S, K, R., et al., Submitted, The Ocular Surface., October 2020). Spontaneous self-touches are the touching movements of the body and face by the hands and fingers, not associated with deliberate decision-making processes and occur approximately 13-23 times per hour—yet despite decades of research, this remains a poorly understood phenomenon and difficult to stop.

The majority of spontaneous self-touches are directed towards the face or the hands and a significant number of face-touches involve the mucosal surfaces of the T-zone. In one study of 26 medical students, mucosal surfaces made up 44% of all face-touches, nearly evenly split between mouth (36%), nose (31%), and eyes (27%), equates to approximately 15% of all spontaneous face-touches involving the ocular mucous membranes, or a rate of 2-3 ocular mucous membrane touches per hour (Spencer, S, K, R., et al., Submitted, The Ocular Surface., October 2020).

There has been late recognition of the likely role of the surface of the eye as the primary invasion site and repurposing of a variety of eye protectors not designed for this purpose. Microbes such as viruses bind the tear film and pass rapidly via the nasolacrimal duct into the nose and thence to invade the nasopharynx. From there infection can spread to the lungs, gut and bloodstream. It was early recognized that the eye and ocular surface may have been the entry point for microbial invasion during the great plagues, respiratory viruses, which are said to exhibit ocular tropism and other bacterial species. Allergens, causing or contributing to hay fever, sinusitis and asthma may also enter via this pathway. In other settings, insects such as flies or mosquitoes attack the face and in certain environments (in this way, the present invention could be seen as an evolution of the Australian cork hat in which suspended corks discourage infestation), fumes (including pepper spray, smoke), dust or other particular matter may make normal activities difficult and even dangerous. Very cold air striking the face and eye can also be injurious.

A variety of other eye protectors, while covering much of the face do not exclude air currents that can circumvent the device. They can obstruct vision (like the original plague masks), fog up, get in the way (particularly when using optical instruments), are uncomfortable (hence liable to diminished or improper use) and when worn as part of a helmet-device, reduce communication with others. The need to exclude an airborne viral invader, preventing aerosol droplets from entering the air mass in front of the eye, has resulted in the use of hermetically sealed eye protection, generally designed for short to medium term usage rather than a shift in a hospital intensive care unit. However, a major problem is limited usability due to the well-known phenomenon of fogging, the main cause of poor compliance (Damn, S. J., Ophthalmic Physiol Opt., 1999; 19:357-61., Chughtai, A. A., et al., Ann Work Expo Health., 2020; 64:368-377.). Condensation occurs on the inner surface of the goggles of evaporating tears, continuously secreted by the ocular adnexae and sweat from the periocular skin into the contained space. Goggles fog significantly by 5 minutes and despite the use of a filtration system, some fogging is present at one hour (Douglas, D., et al., Int J Infect Dis., 2020 June; 95:340-344; and non-provisional patent application pending (U.S. Ser. No. 16/773,960)) and longer term “real world” studies have yet to be carried out. Thus, there are considerable contributing factors limiting efficacy of PPE. This may help in explaining why front-line health care workers who, despite wearing apparently adequate gloves, gowns and face masks are at least a three times increased risk of infection compared to the general population (Nguyen, L. H., et al., Lancet Public Health., 2020; 52468-2667(20)30164-X.).

Extreme measures have been taken to address these issues. Devices such as the BioVYZR 1.0, a ‘Personal Air Purifying Shield’ (VYZR Technologies) have been developed and mimic the attire of astronauts—with cost and mobility limitations. Furthermore, this kind of “blocked-off design” is becoming widespread and being applied architecturally (Haigney, S., The Spectator Australia.2020; 3 October: pp 36-37.).

While the strongest evidence for microbial transmission is via the viral aerosol-oculo-nasal route, the relative importance of this route versus hand auto-inoculation is unknown. While the latter can be addressed by means of good hand hygiene interventions the effectiveness of these interventions can vary greatly. Therefor a reliable means of reducing this infection pathway would be desirable.

Since respiratory infections, viral and bacterial, are often airborne, rather than using physical obstruction (as per conventional PPE) of limited and variable efficacy another approach, used in laboratory and operating room settings, is to use laminar flow of filtered air to prevent contamination—effectively “fighting air with air”. The air-shield device disclosed herein is effectively a light (and comfortably worn), relatively silent transparent shield which cannot become dirty, scratch or fogged. Increasing the flow rate up to high levels will allow its use during fires or where noxious gases such as tear gas are in use.

If it transpires that the eyes are the predominant route of infection, a spectacle design of the air-shield device disclosed herein rather than mechanically blocking air access, may be used as a frame to emit air currents away from the eyes, would be a simpler, lighter version of the concept.

Ultraclean laminar airflow has previously been used to reduce/prevent surgical site infections. If average airborne concentrations of microbe-carrying particles (MCPs)<10/m³, and preferably <1/m³, then deep joint infection after orthopaedic joint replacement is lower than in conventionally ventilated operating rooms (Whyte, W. et al., J Hosp Infect. 2019; 103:e9-e15.). A device known as the SurgiCube®, developed for ophthalmic surgery complies with this higher standard and has been shown to minimize the spread of airborne contamination, including the latest coronavirus SARS-CoV-2 (https://surgicube.com/efficiency-of-surgicube-airflow-device-to-prevent-covid-19-2/accessed 23 Mar. 2020.).

If the hand self-contamination pathway turns out to be a major factor, a means of discouraging the prevalent spontaneous self-touches, will be incorporated in the air-shield device disclosed herein. An infrared or ultrasonic proximity sensor will sense hand approximation to the face and emit a warning—a sound or vibration in the device to warn the wearer that they are about to self-touch and potentially auto-contaminate. (open source devices described but not incorporated in T-zone protective devices: https://medeng.jpl.nasa.gov/covid-19/pulse/#:˜:text=PULSE%20is%20a%203D%2Dprinted,order%20to%20reduce%20potential%20infection.

Furthermore, while deflecting viral-containing aerosols and hands from the main portal(s) of human infection, this would simply redistribute the virus (or other microbial agents) within a particular environment, potentially placing at risk others who also occupy this space. Thus, a viricidal agent which is non-toxic to the T-zone would improve the utility of this device. To this end, personal, portable “air purifiers” have been developed and rely on ion generation to disrupt viral particles. For example, carbon-fiber ionizers produce stable unipolar ions in sufficiently high concentrations with little ozone generation and secondary particles. This has been tested on aerosolized MS2 Bacteriophage, a surrogate for SARS-CoV-2 and it was found that the antiviral efficiency increased with ion exposure time and ion concentration (Hyun, J, et al., J Aerosol Sci., 2017; 107:31-40). The antiviral efficiency increased by 1.7 times when positive and negative air ions existed together (using the bipolar mode ionizer). In the setting of a simulated ICU hospital unit set up to test aerosolised viral particles, a 99% reduction of MS2 bacteriophage was obtained in the air that was ionized (https://blog.plasma-air.com/plasma-air-ionization-proven-to-reduce-coronavirus-surrogate-ms2-bacteriophage-by-99-in-independent-spanish-testing/).

The air-shield device disclosed herein uses laminar air currents, such as currents of clean, filtered, ionized, microbe-free air, as an “air shield” or “halo” to protect the eye-zone or the T-zone of the face, chiefly the eyes (but also nose and mouth) from microbes (or other noxious agents such as insects, dust, allergens, smoke and gases) either air or hand-born. This means of protection is superior to current “brute force” mechanical shields such as masks and goggles by virtue of being lighter, less likely to “leak” (and therefore more effective), more comfortable to wear, with minimal contact with facial structures (and therefore likely to be associated with greater compliance and therefore more effective. Airborne microbes are deflected from T-zone structures and potentially eradicated by ionized air. In addition, a motion detector to warn of subconscious hand to face approximation will warn by vibration, noise or light flash of a potential face touch.

The availability of small, rechargeable, long-acting, lightweight and relatively silent, inexpensive fans allows the possibility of creating a laminar flow device for personal protection. Air intake or egress is via a high-efficiency particulate air (HEPA) filter and is then directed via a partial or complete halo to protect either the face (˜180 degree) or the whole head (360 degrees). Thus, a semi-circular or circular column of filtered air in a laminar column is directed downwards from the laminar airflow device (sometimes referred to as a “halo device”), effectively a vertical laminar flow hood or cylinder for the human face/head.

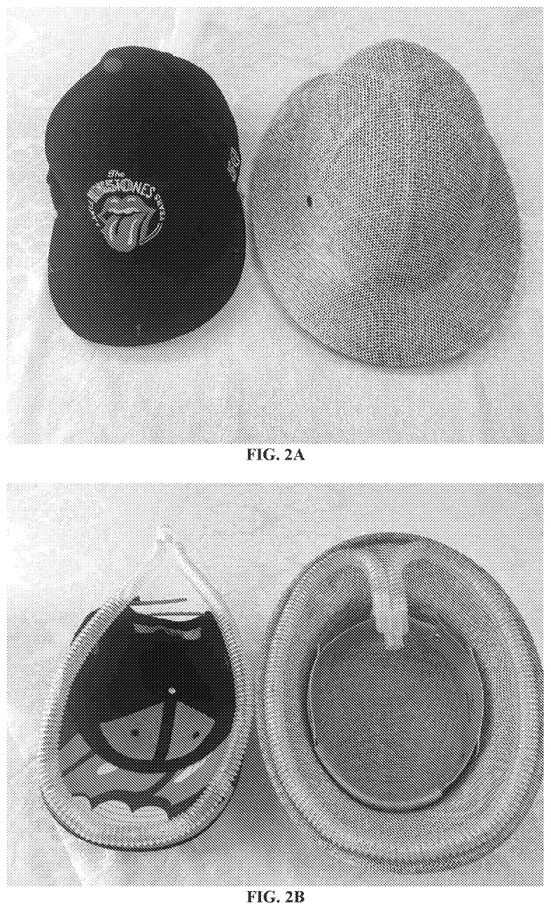

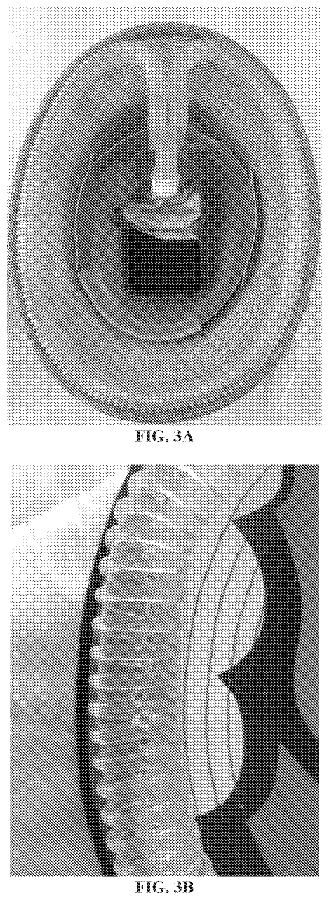

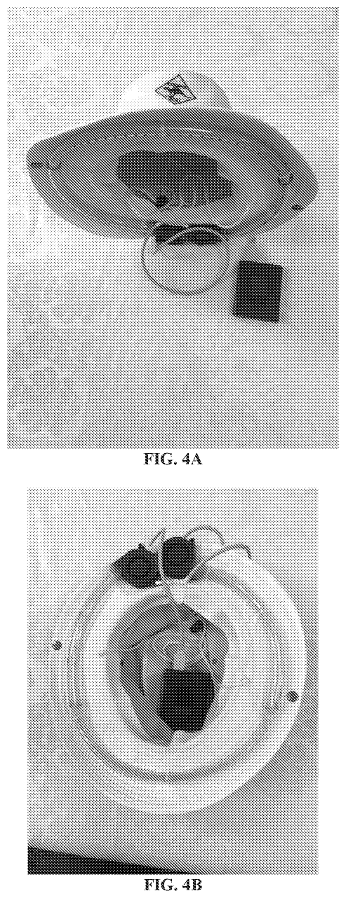

In one embodiment, the air-shield device (halo) includes tubing mounted on the underside of a hat, through 360 degrees or 180 degrees (FIGS. 2A-2B). The tubing has a series of holes placed such the flow of air, driven through the tubing exits in a downward direction and in a laminar pattern. The fan and a HEPA filter can be mounted in the peak of the hat (FIGS. 3A-3B and 4A-4B) or remotely, such as clipped to the waist on a belt and connected to the hat/halo via tubing. The fan or fans are light-weight, rechargeable, with a long battery life and may include charging by a solar panel (mounted on the hat). As in FIGS. 4A-4B, multiple fans can be fitted along the rim of the hat, discharging eye directly rather than through tubing.

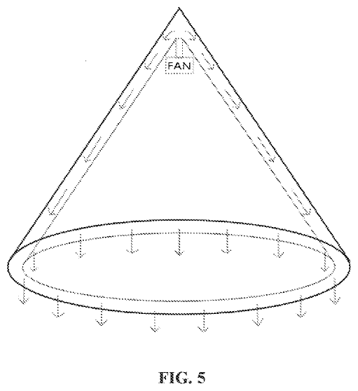

Another embodiment includes a “hat within a hat” (FIG. 5; head-covering within a head-covering) to allow emission of a column of air through 360 degrees.

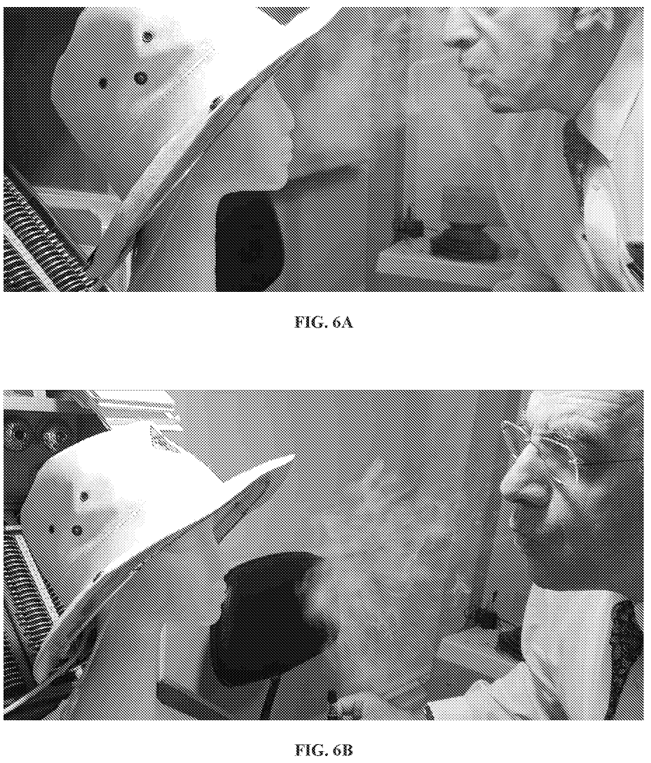

In initial studies, the air-shield device disclosed herein has been shown to effectively prevent smoke generated by either an e-cigarette or a fog machine from the “face” of a mannequin or human wearing the device with the fan switched on (se FIGS. 6A-6B).

Role of the Convective Boundary Layer

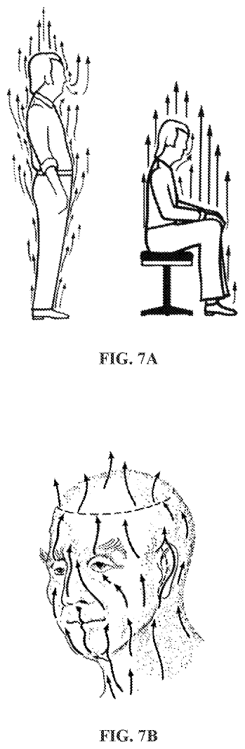

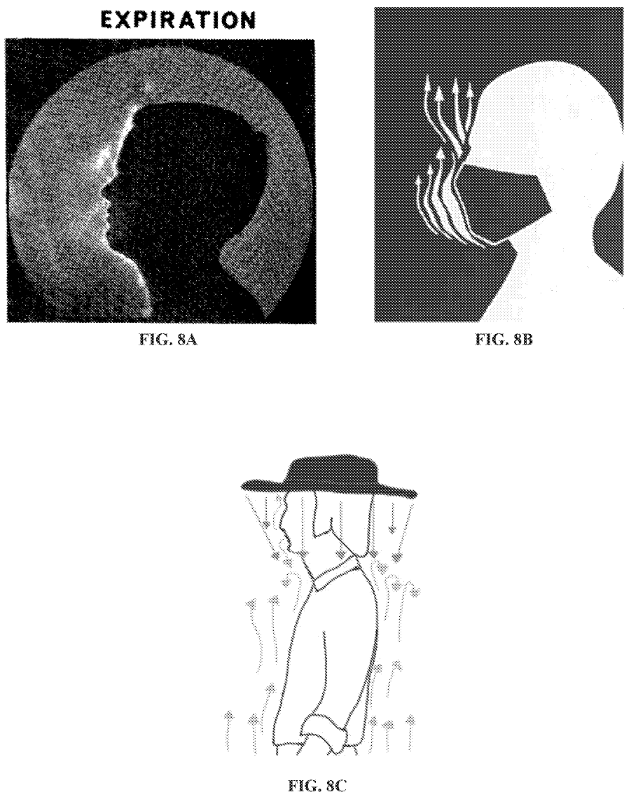

Another critical element relates to the concept of the convective boundary layer (CBL) enveloping the human body and particularly the face and eyes (FIGS. 7A-7B). Convective heat loss, caused by the temperature gradient between a human body surface (clothed or nude) and cooler surrounding air, induces upward natural flow (from feet to head—FIG. 7A) of the surrounding air, which rises above the head and forms a human thermal plume in built environments (Lewis, H. E. et al., Lancet., 1969; 1(7609):1273-7; Clark, R. P., et al. J R Soc Interface., 2009; 6 (Suppl 6):S767-82). For this reason, smokers hold cigarettes at some distance away, to stop smoke from reaching their eyes (Hyldgard, C-E., Indoor Environmental Technology., 1994; Vol. R9414 No. 39:413-433.). This layer is modified by facial contours (FIG. 7B) with air flowing over the cheeks to the eyes and pre-orbital regions. The boundary layer thickens in front of the eye from the 1-2 cm normal envelope thickness and travels at a speed of ˜0.5M/sec. As well, air exhaled through the nose usually becomes part of the convective flow around the body (Hyldgard, C-E., 1994) and perhaps accounting for the thickening of pre-ocular boundary layer, which does not appear to extend further than the tip of the nose (FIG. 8A). This exhaled air provides some moisture and likely microbiome elements to the surface of the eye and it has been said in Japan, where facemask wearing is prevalent, that “mask is an excellent way of directing moisture toward the eyes, especially when the air is dry or there is frost . . . the main reason why [she] individuals wear[s] a mask: to ‘give [her] the eyes a bath’” (Dexter G., The Spectator Australia.2020; 19 September: pp 17.).) This redirection of air by a mask is seen in FIG. 8B.

For these reasons, the air-shield device disclosed herein is positioned such that the downward laminar air current is about 3 cm, for example greater than 3 cm, from the forehead, and with a brimmed hat-based device the tubing may be placed out to 7.5 cm from the forehead (FIG. 8C). This minimizes interference with the physiological CBL updraft, interference with which may cause turbulence. If the downdraft is placed closer than about 3 cm, this may cause discomfort by drying of the tear film of the subject wearing the air-shield device. In some embodiments, air from the peripheral rim of the air-shield device is aimed at the area of the chin, to deflect the normal (potentially viral laden air) from the lower part of the body. This is in addition to a vertical current of air place ˜3 cm from the surface of the eye. See FIG. 8C, outermost arrows pointing inward from the air-shield device towards the subject.

In some embodiments, the airflow rate emitted from the air-shield device is adjustable, for example, at an air speed of about 0.425 m/s, for efficient particle transportation, which has been shown to offer protection against pollutants in a room environment (Licina, D., et al., Indoor Air., 2015; 25:21-35.). While there is potential for thermal discomfort, the air stream can be either placed far enough away from the face for this to be negligible and/or the temperature of the air can be controlled with a temperature element. In cases of extreme environmental exposure such as firefighters exposed to smoke, air speeds that are used in fume hoods (National Research Council (US) Committee on Prudent Practices in the Laboratory. Prudent Practices in the Laboratory: Handling and Management of Chemical Hazards: Updated Version. Washington (DC): National Academies Press (US); 2011. 9, Laboratory Facilities. Available from: https://www.ncbi.nlm.nih.gov/books/NBK55867/) of about 0.4-0.5 m/s will be achievable with a range up to 5 m/s.

Spectacle Device

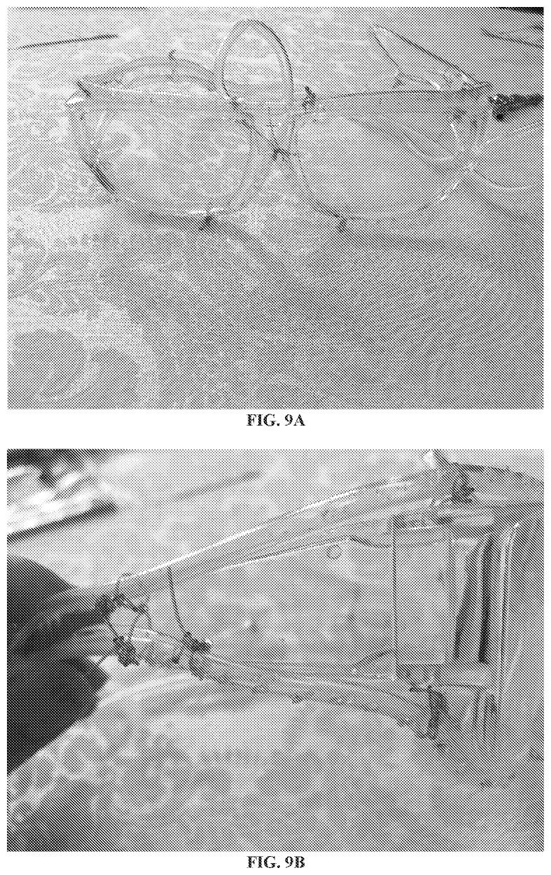

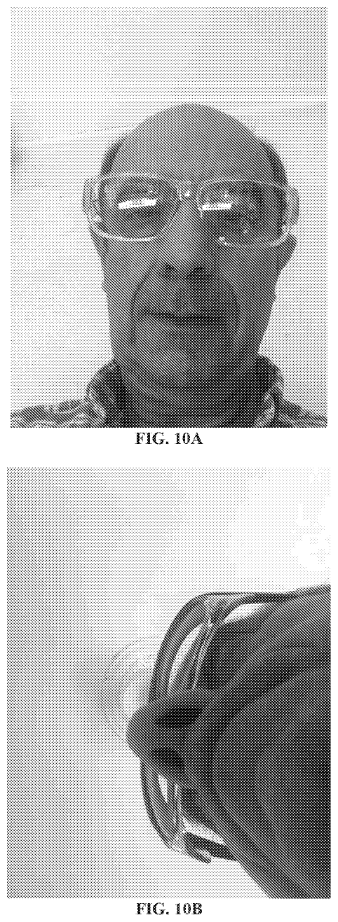

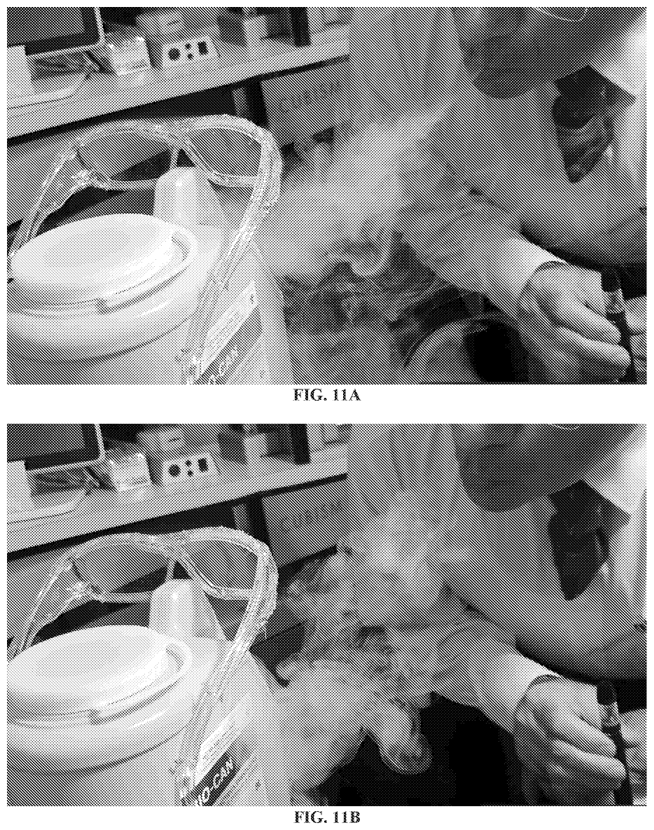

Since the likely predominant access point for viral invasion of humans is via the eye and tear film (Coroneo, M. T., Ocul Surf., 2020:S1542 0124(20)30089-6.) and there is evidence that even the wearing of conventional spectacles offers some protection (Zeng, W., et al., JAMA Ophthalmol. Published online Sep. 11, 2020. Jdoi:10.1001/jamaophthalmol.2020.3909., Maragakis, LL., JAMA Ophthalmol., 2019; Published online Sep. 16, 2020), the development of improved eye protection has priority. By using a spectacle frame silhouetted with perforated tubing, laminar airflow is directed away from the spectacle frame and the eyes (FIG. 9A, B). In particular, this airflow “covers” the lateral and inferior aspects of the frame, which are normally open to air directed from the sides and below (FIGS. 10A-10B). Given the variability of human facial morphology, it is virtually impossible to design spectacles to block air access, without resorting to a goggle design, with its disadvantages. By using “air cover”, fogging is avoided and these exposed zones re protected by the air shield (FIGS. 11A-11B). Furthermore, air can be diverted behind the air shield onto the posterior aspects of the spectacle frame to further minimize the risk of fogging. Additionally, lenses can be left out of the frame as sufficient air can directed in a forward and downwards direction to prevent oncoming air from striking the face.

In some embodiments, the composition of the air laminar emitted from the air-shield device disclosed herein may be controlled so that, apart from excluding particulate matter and microorganisms, the temperature may be cooled, heated, or the humidity adjusted to counter adverse climatic conditions. Thus, for example in dry eye syndrome (Coroneo, M., Medicine Today 2013; 14: 53-61.), the air can be humidified to provide a periocular moist air environment. Attempts to do this with tight-fitting spectacles (Shen, G., et al., Optom Vis Sci., 2016; 93:158-164) or devices that deliver moisture (Trevor-Roper, P. D., The Frank Flynn tear-supplying spectacles. Trans Ophthalmol Soc U K., 1967; 87:105-7.) to the surface of the eye have limitations such as fogging, are of limited usefulness and not widely used.

In some embodiments, the emitted laminar air humidity may be controlled with a second fan system releasing air directed at the eyes “inside” outer protective cylinder of air. This second fan system may also use filtered air passed through a moisturising system. The open nature of the system (without lenses, would mean that fogging is impossible and they eyes are both protected and kept moist, simultaneously (Moshirfar, M., et al., Ophthalmol Ther. 2020; 9:397-400., Amador, G. J., et al., J R Soc Interface. 2015; 12:20141294., Ogawa, M., et al. Eye Contact Lens., 2018; 44:379-383.).

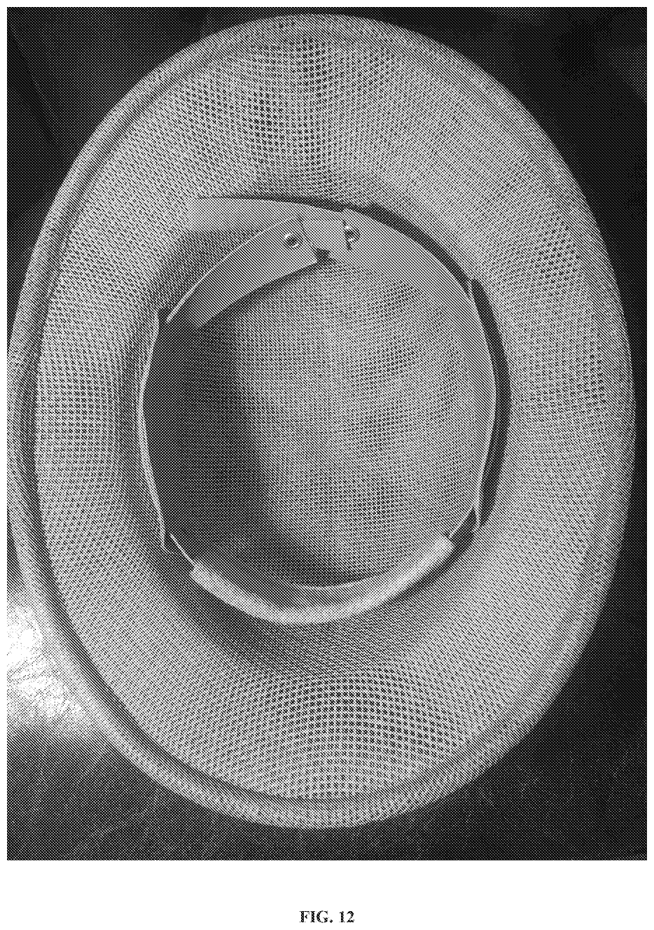

In some embodiments, the air-shield device disclosed herein is comfortable to wear, which provides an advantage over many currents PPE devices. For example, key elements of hat/headgear design are comfort (Ball, R. M., 2011. Human Factors in Protective Headgear Design. In: Human Factors and ergonomics in Consumer Products: Uses and Applications, Chapter 19. Editors: Karwoski, W, Soares, M. Taylor and Francis pp. 302-313, 2011., Bogerd, C. P., et al., International Journal of Industrial Ergonomics. 2015; 45:1-12.), a challenge given the multiplicity of head shapes and environments and appearance. Three key elements will be addressed in the design of the air-shield device disclosed herein: (1) temperature—air from the fan will be diverted into the space between the hat and head to allow for cooling or warming, as required. A separation of hat from head by the use of an “internal” headband (FIG. 12). Apart from allowing space for air to circulate, this effectively minimizes contact between head and hat as in Asian conical hats for which contact is minimized by the tangential point of contact. (2) The air-shield device disclosed herein may come as an integrated system or may be capable of being retro-fitted into an existing hat design, such as the popular and ubiquitous baseball hat (FIG. 2A) or into an existing spectacle design. (3) The air-shield device must be lightweight: achieved by the use of light, efficient, relatively silent fans with power sources that can be “onboard”, including a solar panel or separated—either attachable to a belt or a pocket to an external power source (as for example in an operating room setting).

Ion Generator Component

A concern in relation to any virus deflecting device is that it redistributes the virus, particularly in environments with poor ventilation. To address this issue, an ion generator may be incorporated in the air-shield device disclosed herein, so that ionised air may be emitted by the device. Ionization of air may be used for “air cleaning” and may result in “increasing psychological health, productivity, and overall well-being but without consistent or reliable evidence in therapeutic effects and with controversy in anti-microorganisms” (Jiang, S. Y., et al., Int J Mol Sci., 2018; 19:2966.). However, ionizer devices have been shown to reduce infectivity of influenza viruses and to reduce transmission of Newcastle disease virus and several kinds of bacteria in animal experiments and to eliminate allergens from the air (Hagbom, M., et al., Sci Rep. 2015; 5:11431.). Recently, plasma air ionization technology has been effective in the reduction of MS2 Bacteriophage, a surrogate for SARS-CoV-2 (COVID-19), in indoor environments (https://www.prnewswire.com/news-releases/plasma-air-ionization-proven-to-reduce-coronavirus-surrogate-ms2-bacteriophage-by-99-in-independent-spanish-testing-301076955.html).

In some embodiments, the air-shield device disclosed herein may further comprise an ionizer, such as a carbon-fiber ionizer, capable of generating positive or negative ions. Bipolar ion treatment has been reported as being more effective by about 1.7 times than unipolar ion in anti-viral efficacy (Hyun, J., et al., J Aerosol Sci., 2017; 107:31-40; also see Philips air purifier IP; WO/2005/087320).

Movement Detector to Warn Hands are Going Near Face

Hand hygiene with alcohol-based hand rub is widely used around the world as one of the most effective, simple and low-cost procedures against COVID-19 cross-transmission (WHO. Infection prevention and control during health care when novel coronavirus (nCoV) infection is suspected. Interim guidance. WHO, 25 Jan. 2020 hutps://apps.who.intlirisirest/bitstrearns112662961retrieve, Spencer, S, K, R., et al., Submitted, The Ocular Surface., October 2020). One reason for the effectiveness of this strategy may relate to spontaneous face-touching in humans as indicated, occurring 17-23 times per hour (6,7) with about 50% of half of spontaneous face-touches involve mucous membranes and about 30% one third of these involving the eyes. Eye rubbing is also considered a contributing factor in a condition known as keratoconus in which corneal warpage reduces vision (Najmi, H., et al., Int J Ophthalmol., 2019; 12:1775-1781.) and which may require corrective surgical intervention. Current methods of preventing eye rubbing are largely ineffective. Eye rubbing may also play a role in allergic eyelid dermatitis (Chisholm, S. A. M., et al., Ophthalmic Plast Reconstr Surg., 2017; 33:248-250.).

In some embodiments, if the hand self-contamination pathway turns out to be a major factor, a means of discouraging the prevalent spontaneous self-touches, will be incorporated in the air-shield device disclosed herein. In some embodiments, the air-shield device disclosed herein may further comprise an infrared or ultrasonic proximity sensor to sense hand approximation to the face and emit a warning (a sound or vibration in the device to warn the wearer that they are about to self-touch and potentially auto-contaminate). For example, see: https://medeng.jpl.nasa.gov/covid-19/pulse/#:˜:text=PULSE%20is%20a%203D%2Dprinted,order%20to%20reduce%20potential%20infection.

In some embodiments, the air-shield device disclosed herein, and methods of using the same, is primarily designed to prevent airborne noxious agents such as viruses, other micro-organisms, allergens, chemicals, including gases from reaching the human face and in particular the ocular and periocular surface. This will reduce the risk of systemic invasion via pathways such as the ocular surface—nasolacrimal-nasopharyngeal pathway (Coroneo, M. T., Ocul Surf., 2020:S1542-0124(20)30089-6.). In some embodiments, the air-shield device disclosed herein is more effective form of PPE than the current traditional forms. In some embodiments, the air-shield device disclosed herein, and methods of using the same, may play a role in limiting the spread of contagious diseases and also in the management of allergy and eye conditions such as keratoconus.

In some embodiments, an air-shield device is provided comprising: i) an airflow-generating device; and ii) a laminar airflow device having a vent or a plurality of exit ports, wherein the laminar airflow device is in communication with the airflow-generating device; wherein the air-shield device is a personal protection air-shield device. In some embodiments, the personal protection air-shield device is a personal eye-zone protection air-shield device or is a personal T-zone protection air-shield device.

In some embodiments, the airflow-generating device comprises a fan or a plurality of fans. In some embodiments, the airflow-generating device is electric powered, battery powered, rechargeable battery powered, and/or solar powered. In some embodiments, the airflow-generating device further comprises an air-intake port and an air-output port. In some embodiments, the laminar airflow device is flexible and/or mouldable.

In some embodiments, the air-shield device further comprises an air cleaning device. For example, in some embodiments, the airflow-generating device is connected to an air cleaning device via the air-intake port of said airflow-generating device. In some embodiments, the air-intake port is configured with an air cleaning device, for example, the air cleaning device may be positioned and configured for transferring a flow of cleaned air through the airflow-generating device. In some embodiments, the air cleaning device comprises an air cleaning screen, a UVC device, and/or an air cleaning filter. In some embodiments, the air cleaning filter is a HEPA filter. In some embodiments, the air cleaning filter excludes (filters) an aerosolized droplet, an airborne agent, and/or an airborne particle. In some embodiments, the air cleaning filter excludes (filters) an airborne particle having a diameter of about 0.01-0.20 um or greater, such as particles having a diameter of about 0.05-0.15 um or greater. In some embodiments, the cleaned air flowing from the air cleaning device is exclusive of particles having a diameter of about 0.01-0.20 um or greater, such as particles having a diameter of about 0.05-0.15 um or greater. In some embodiments, the cleaned air flowing through the airflow-generating device is exclusive of particles having a diameter of about 0.01-0.20 um or greater, such as particles having a diameter of about 0.05-0.15 um or greater. In some embodiments, the cleaned air flowing through the laminar airflow device is exclusive of particles having a diameter of about 0.01-0.20 um or greater, such as particles having a diameter of about 0.05-0.15 um or greater.

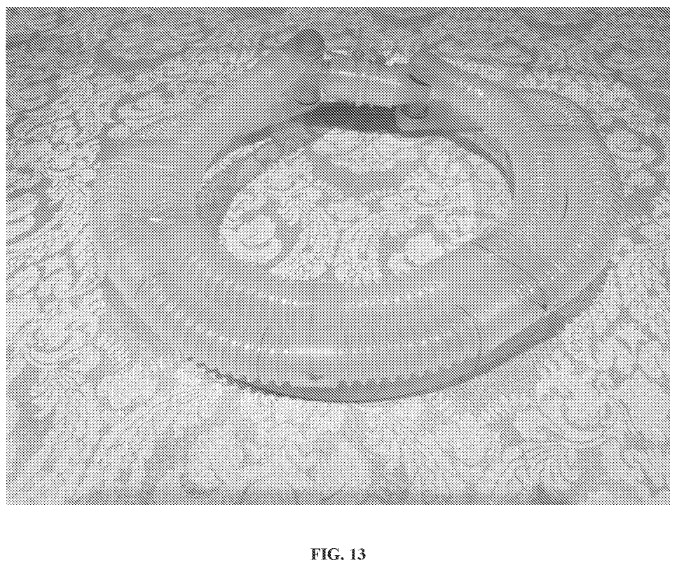

In some embodiments, the air-shield device comprises a plurality of laminar airflow devices. For example, in some embodiments, the air-shield device comprises a first laminar airflow device and a second laminar airflow device, wherein each of the first and the second laminar airflow devices have a vent or a plurality of exit ports, wherein each of the first and the second laminar airflow devices is in communication with the airflow-generating device; wherein the air-shield device is a personal protection air-shield device. In some embodiments, one or more of the plurality of laminar airflow devices has a vent or a plurality of exit ports angled in such a manner as to direct the airflow coplanar with the forehead, face, side, and/or back of the head of a subject wearing the air-shield device, and one or more of the plurality of laminar airflow devices has a vent or a plurality of exit ports angled in such a manner as to direct the airflow angled towards the chin and or the base of the head of the subject wearing the air-shield device. For example, in some embodiments, the air-shield device comprises two laminar airflow devices (or a single laminar airflow device having two separate tubes, such as is shown in FIG. 13)), with a first laminar airflow device (or first tube) positioned more proximate to the head of the subject wearing the air-shield device (sometimes referred to as an inner laminar airflow device) having a vent or a plurality of exit ports angled in such a manner as to direct the airflow coplanar with the forehead, face, side, and/or back of the head of a subject wearing the air-shield device, and a second laminar airflow device (or second tube) positioned more distal to the head of the subject wearing the air-shield device (sometimes referred to as an outer laminar airflow device) having a vent or a plurality of exit ports angled in such a manner as to direct the airflow angled towards the chin and or the base of the head of the subject wearing the air-shield device. See, e.g., FIG. 8C. In some embodiments, the air-shield device comprising two laminar airflow devices (or a single laminar airflow device having two separate tubes, such as is shown in FIG. 13)), the first laminar airflow device (or first tube, or inner laminar airflow device) has a vent or a plurality of exit ports angled in such a manner as to direct the airflow angled towards the chin and or the base of the head of the subject wearing the air-shield device, and the second laminar airflow device (or second tube, or outer laminar airflow device) has a vent or a plurality of exit ports angled in such a manner as to direct the airflow coplanar with the forehead, face, side, and/or back of the head of a subject wearing the air-shield device.

In some embodiments, the laminar airflow device emits cleaned, filtered air from the airflow-generating device and through the vent or the plurality of exit ports. The laminar airflow device may be connected to said airflow-generating device via the air-output port. The laminar airflow device, for example, may be a tube, such as a plastic tube. In some embodiments, the laminar airflow device is a tube, wherein at least one end of the tube is connected to and in communication with the airflow-generating device, wherein both ends of the tube are connected to and in communication with the airflow-generating device, or wherein one end of the tube is connected to and in communication with the airflow-generating device and the second end of the tube is a sealed closed end. In some embodiments, the tube has an inner diameter of at least 1 mm, 2 mm, 3 mm, 4 mm, 5 mm, 6 mm, 7 mm, 8 mm, 9 mm, 10 mm, or 15 mm. In some embodiments, the tube has an inner diameter of less than 10 cm. In some embodiments, the tube has an outer diameter of at least 1 mm, 2 mm, 3 mm, 4 mm, 5 mm, 6 mm, 7 mm, 8 mm, 9 mm, 10 mm, or 15 mm. In some embodiments, the tube has an outer diameter of less than 10 cm.

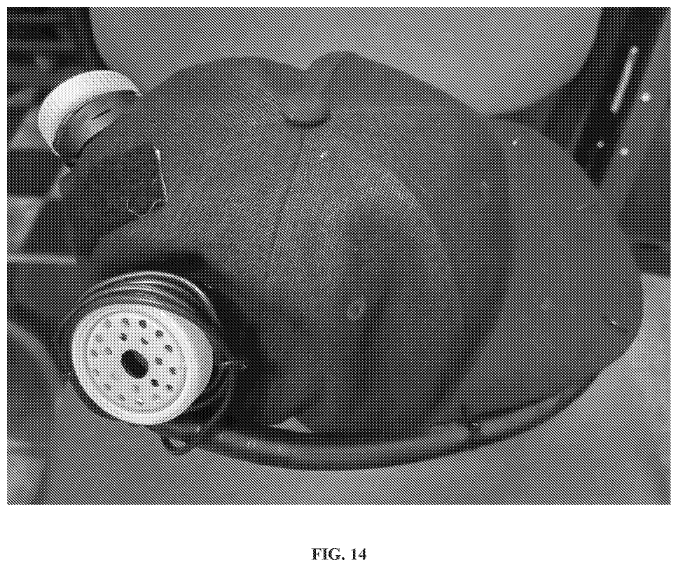

In some embodiments, the laminar airflow device comprises one or more air filtration systems, said air filtration systems each comprising one or more air filters (HEPA). See FIG. 14. In some embodiments, the air filters of the air filtration system are housed in chambers. In some embodiments, the air filtration system filters and allows air to flow to the laminar airflow device (tubing attached to the underside of the rim). In some embodiments, an air filtration system functions as an air intake for the laminar airflow device. In some embodiments, an air filtration system filters and allows air to vent from the laminar airflow device. In some embodiments, an air filtrations system is situated between the inlets and vents of the laminar airflow device.

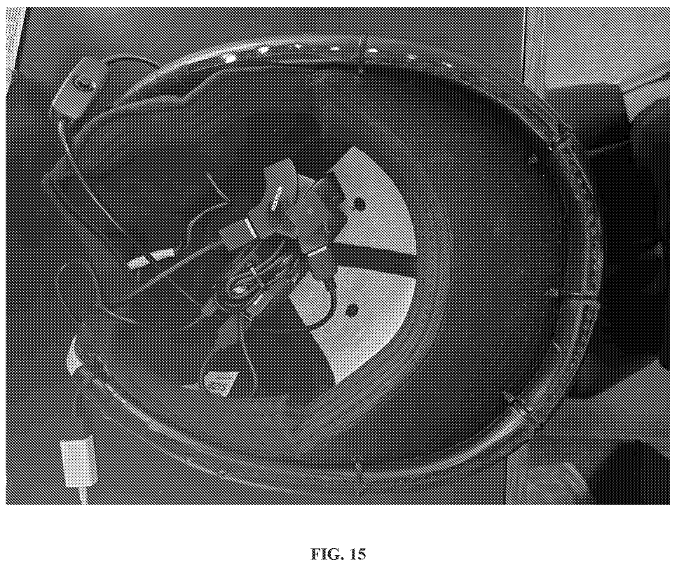

In some embodiments, the laminary airflow device comprises a light-emitting device (light source). See FIG. 15. In some embodiments, a light source may emit one or more wavelengths of light. In some embodiments, the light source emits one or more wavelengths of light that exclude UVC light. In some embodiments, the light source emits one or more wavelengths of light that include UVC light (UVC device). In some embodiments, a UVC device is an LED strip. In some embodiments, the UVC light has a wavelength greater than 280 nm. In some embodiments, the UVC light has a wavelength less than 395 nm. In some embodiments, the UVC light has a wavelength between 280 nm and 395 nm. In some embodiments, the UVC light has a range of wavelengths between 280 nm and 395 nm. In some embodiments, a UVC device is situated at the inlet of the laminar airflow device. In some embodiments, a UVC device is inside the tubing of the laminar airflow device. Said tubing may be opaque to UVC radiation, so that the UVC dosage received by the subject wearing the laminary airflow device is reduced. In some embodiments, the UVC device may also emit visible light. For example, the UVC device might emit blue light. In some embodiments, the UVC device emits UVA light. In some embodiments, the UVC device emits UVB light.

In some embodiments, a portion of the laminar airflow device is exclusive of the vent or the plurality of exit ports. In some embodiments, the laminar airflow device emits laminar airflow through the vent or the plurality of exit ports. The vent, for example, may define an opening through which laminar air is emitted from the laminar airflow device. In some embodiments, each of the plurality of exit ports defines an opening through which laminar air is emitted from the laminar airflow device. In some embodiments, one or more exit ports of the plurality of exit ports has a circular perimeter, for example, the opening of one or more exit ports of the plurality of exit ports has a circular perimeter. In some embodiments, one or more exit ports of the plurality of exit ports has an oblong perimeter, for example, the opening of one or more exit ports of the plurality of exit ports has an oblong perimeter. In some embodiments, one or more exit ports of the plurality of exit ports is a vent, or the plurality of exit ports comprises a series of vents. In some embodiments, the opening of one or more exit ports of the plurality of exit ports is a slit-shaped vent. In some embodiments, the plurality of exit ports comprises at least a first series of exit ports and a second series of exit ports, for example, the at least first series of exit ports and second series of exit ports are positioned to minimize or avoid gaps of emitted laminar airflow.

In some embodiments, one or more exit ports of the plurality of exit ports has a diameter of at least 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 3 mm, 4 mm, or 5 mm. In some embodiments, the vent has a width of at least 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 3 mm, 4 mm, or 5 mm, wherein the width is measured along the perimeter of said tube. In some embodiments, the vent has a length of at least 0.2 cm, 0.3 cm, 0.4 cm, 0.5 cm, 1 cm, 2 cm, 3 cm, 4 cm, 5 cm, 6 cm, 7 cm, 8 cm, 9 cm, or 10 cm, wherein the length is measured along the cylindrical length of the tube. In some embodiments, the vent defines a slit-shaped opening traversing along the cylindrical length of the tube. For example, the vent is a slit-shaped vent, such as a slit-shaped vent traversing along the cylindrical length of the tube. In some embodiments, the slit-shaped vent defines a slit-shaped opening traversing along the cylindrical length of the tube.

In some embodiments, laminar airflow is emitted through the vent or the plurality of exit ports to create a shield of laminar air about the head of the subject wearing said personal protection air-shield device. In some embodiments, laminar airflow is emitted through the vent or the plurality of exit ports to create a shield of laminar air about the face of the subject wearing said personal protection air-shield device. For example, the vent or the plurality of exit ports direct the current of the emitted laminar airflow at a position about 3 cm relative to the forehead, face, or side of the subject wearing said personal protection air-shield device, such as at a position about 3 cm relative to the forehead, face, side, or back of the head of the subject wearing said personal protection air-shield device. In some embodiments, the vent or the plurality of exit ports to direct the current of the emitted laminar airflow at a position at least 3 cm, at least 4 cm, at least 5 cm, at least 6 cm, or at least 7 cm, relative to the forehead, face, or side of the head of the subject wearing said personal protection air-shield device, such as at a position at least 3 cm, at least 4 cm, at least 5 cm, at least 6 cm, or at least 7 cm, relative to the forehead, face, side, or back of the head of the subject wearing said personal protection air-shield device.

In some embodiments, the emitted laminar airflow is a stream of laminar air or a plurality of streams of laminar air. In some embodiments, the emitted laminar airflow is a plane of laminar air or a plurality of parallel planes of laminar air. For example, the emitted laminar airflow may be about the face of the head of the subject wearing said personal protection air-shield device. In some embodiments, the emitted laminar airflow is proximate to and about co-planar with the face and sides of the head of the subject wearing said personal protection air-shield device, wherein the emitted laminar airflow is about the circumference of the head of the subject wearing said personal protection air-shield device. In some embodiments, the emitted laminar airflow spans at least 180 degrees about the circumference of the head of the subject wearing said personal protection air-shield device, such as spans at least 180 degrees, 200 degrees, 220 degrees, 240 degrees, 260 degrees, 280 degrees, 300 degrees, 320 degrees, or 340 degrees about the circumference of the head of the subject wearing said personal protection air-shield device. In some embodiments, the emitted laminar airflow spans between 180 degrees and 360 degrees about the circumference of the head of the subject wearing said personal protection air-shield device, such as spans 360 degrees about the circumference of the head of the subject wearing said personal protection air-shield device. In some embodiments, the circumference of the head of the subject wearing said personal protection air-shield device is measured about the cylindrical perimeter comprising both ears and nose of said subject.

In some embodiments, the rate of the laminar airflow is a constant rate or an adjustable rate. For example, the laminar airflow may be emitted at a rate in the range of about 0.3-0.7 m/s, 0.3-0.5 m/s, 0.3-0.4 m/s, or 0.4-0.5 m/s, such as emitted at a rate of about 0.3 m/s, 0.4 m/s, 0.425 m/s, or 0.5 m/s. In some embodiments, the volume of the laminar air emitted from the air-shield device is a constant volume or an adjustable volume. For example, the laminar air emitted from the air-shield device may have a volume in the range of about 0.3-0.7 m³/s, 0.3-0.5 m³/s, or 0.4-0.6 m³/s, such as a volume of about 0.3 m³/s, 0.4 m³/s, 0.5 m³/s, 0.6 m³/s, or 0.7 m³/s.

In some embodiments, the personal protection air-shield device further comprises a head-covering or a spectacle device. In some embodiments, the personal protection air-shield device is worn, or capable of being worn, by a subject exclusive of a head-covering or a spectacle device. For example, in some embodiments, the laminar airflow device is worn directly, or capable of being worn directly, on the head of the subject exclusive of a head-covering or a spectacle device. In some embodiments, the airflow-generating device is attachable to a head-covering. In some embodiments, the personal protection air-shield device comprises the head-covering. For example, in some embodiments, the airflow-generating device is attached to the head-covering or is positioned in the range of between 0.001-1 m from the head-covering. In some embodiments, the laminar airflow device is mouldable to adopt the shape of the head-covering, is mouldable to adopt the shape of an edge of the head-covering, is mouldable to traverse along an edge of the head-covering, is mouldable to traverse along the perimeter of the head-covering, is mouldable to adopt the shape of the perimeter of the head-covering, and/or is positioned about the circumference of the head-covering. In some embodiments, the laminar airflow device is attachable to the head-covering. In some embodiments, the laminar airflow device is attached to the head-covering, such as attached or mounted to an edge of the head-covering, for example, attached or mounted to the underside of the head-covering or attached or mounted to the underside of a brim of the head-covering.

In some embodiments, the laminar airflow device connected to the head covering is positioned to direct the emitted airflow through the vent or the plurality of exit ports at about 3 cm relative to the forehead, face, side, or back of the head of the subject wearing said personal protection air-shield device, for example, positioned to direct emitted airflow through the vent or the plurality of exit ports at least 3 cm, at least 4 cm, at least 5 cm, at least 6 cm, or at least 7 cm, relative to the forehead, face, side, or back of the head of the subject wearing said personal protection air-shield device. In some embodiments, the vent has a length extending at 10%, 20%, 30%, 40%, 50%, 60%, 70%, 80%, 90%, or 100% the circumference of the head-covering, wherein the circumference of the head-covering is measured along an edge of said head-covering. In some embodiments, the vent has a length extending at least 10%, 20%, 30%, 40%, 50%, 60%, 70%, 80%, or 90%, of the circumference of the head of the subject wearing said personal protection air-shield device, for example the vent has a length extending 100% of the circumference of the head of the subject wearing said personal protection air-shield device.

In some embodiments, the circumference of the head of the subject wearing said personal protection air-shield device is measured about the cylindrical perimeter comprising both ears and nose of said subject. The emitted laminar airflow may have a trajectory in the direction from the head towards the feet of the subject wearing said personal protection air-shield device comprising or connected to the head-covering. In some embodiments, the emitted laminar airflow is about parallel to the forehead, face, side, or back of the head of the subject wearing said personal protection air-shield device comprising or connected to the head-covering, such as about 1-15 degrees from parallel to the forehead, face, side, or back of the head of the subject wearing said personal protection air-shield device comprising or connected to the head-covering. In some embodiments, the emitted laminar airflow is angled away from the forehead, face, side, or back of the head of the subject wearing said personal protection air-shield device comprising or connected to the head-covering, such as angled about 1-15 degrees from parallel away from the forehead, face, side, or back of the head of the subject wearing said personal protection air-shield device comprising or connected to the head-covering. In some embodiments, the laminar airflow emitted from laminar airflow device is at least 3 cm, at least 4 cm, at least 5 cm, at least 6 cm, or at least 7 cm, from the forehead, face, side, or back of the head of the subject wearing said personal protection air-shield device comprising or connected to the head-covering. In some embodiments, the laminar airflow emitted from laminar airflow device is at least 3 cm, at least 4 cm, at least 5 cm, at least 6 cm, or at least 7 cm, from the forehead, upper face, upper side, or upper back of the head of the subject, and angled towards the chin and/or base of the head, wearing said personal protection air-shield device. In some embodiments, the personal protection air-shield device further comprises a head-covering.

In some embodiments, the head-covering is fixable upon the head of a subject. In some embodiments, the head-covering is exclusive of a full head-encapsulating head-covering, such as exclusive of a hermetically sealed head-covering. In some embodiments, the head-covering has a brim. In some embodiments, the head-covering is a hat, a helmet, or head garmet, for example the hat is a brimmed hat, such as a baseball hat or a safari hat. In some embodiments, the head-covering directs current of the airflow emitted from the laminar airflow device at least 2 cm, at least 3 cm, at least 4 cm, at least 5 cm, at least 6 cm, or at least 7 cm, relative to the forehead, face, side, or back of the head of the subject wearing said personal protection air-shield device.

In some embodiments, the personal protection air-shield device comprises the spectacle device. In some embodiments, the airflow-generating device is attachable to a spectacle device or is attached to the spectacle device. In some embodiments, the airflow-generating device is positioned in the range of between 0.001-1 m from the spectacle device. In some embodiments, the laminar airflow device is mouldable to adopt the shape of the spectacle frame, is attachable to the spectacle device, and/or is attached to the spectacle device. In some embodiments, the laminar airflow device has a spectacle frame shape, is a spectacle frame, is a spectacle frame housing the laminar airflow device, is a spectacle frame, or is a spectacle frame-shaped laminar airflow device.

In some embodiments, the laminar airflow device connected to the spectacle device is positioned to direct the emitted airflow through the vent or the plurality of exit ports at about 3 cm relative to the forehead, face, or side of the subject wearing said personal protection air-shield device, such as positioned to direct emitted airflow through the vent or the plurality of exit ports at least 3 cm, at least 4 cm, at least 5 cm, at least 6 cm, or at least 7 cm, relative to the forehead, face, or side of the subject wearing said personal protection air-shield device. In some embodiments, the vent has a length extending at least 10%, 20%, 30%, 40%, 50%, 60%, 70%, 80%, or 90%, of the spectacle device, such as a length extending 100% of the spectacle device. In some embodiments, the emitted laminar airflow is about parallel to the forehead, face, or side of the subject wearing said personal protection air-shield device comprising or connected to the spectacle device. For example, the emitted laminar airflow may be about 1-15 degrees from parallel to the forehead, face, or side of the subject wearing said personal protection air-shield device comprising or connected to the spectacle device, such as angled away from the forehead, face, or side of the subject wearing said personal protection air-shield device comprising or connected to the spectacle device, for example, angled about 1-15 degrees from parallel away from the forehead, face, or side of the subject wearing said personal protection air-shield device comprising or connected to the spectacle device. In some embodiments, the laminar airflow emitted from laminar airflow device is at least 3 cm, at least 4 cm, at least 5 cm, at least 6 cm, or at least 7 cm, from the forehead, face, or side of the subject wearing said personal protection air-shield device comprising or connected to the spectacle device.

In some embodiments, the personal protection air-shield device further comprises an air-temperature controlling device. For example, the air-temperature controlling device may be positioned proximate to the air-intake port, or the air-temperature controlling device is positioned proximate to the air-output port. In some embodiments, the air-temperature controlling device is an air-heating device. In some embodiments, the emitted laminar airflow has a higher temperature relative to the temperature of the air entering the air-intake port. In some embodiments, the air-temperature controlling device is an air-cooling device. In some embodiments, the emitted laminar airflow has a lower temperature relative to the temperature of the air entering the air-intake port.

In some embodiments, the personal protection air-shield device further comprises an air-humidity controlling device. The air-humidity controlling device may be positioned proximate to the air-intake port or may be positioned proximate to the air-output port. In some embodiments, the emitted laminar airflow has more humidity relative to the humidity of the air entering the air-intake port, or the emitted laminar airflow has less humidity relative to the humidity of the air entering the air-intake port. In some embodiments, the air-humidity controlling device comprises a fan system.

In some embodiments, the personal protection air-shield device further comprises an air-ionizing device. For example, the air-ionizing device may be positioned proximate to the air-intake port or may be positioned proximate to the air-output port. In some embodiments, the air-ionizing device is a carbon-fiber ionizer. In some embodiments, the emitted laminar airflow comprises ionized air. In some embodiments, the personal protection air-shield device emits, or is capable of emitting, clean, ionized, laminar air.

In some embodiments, the personal protection air-shield device further comprises a proximity-sensor device, for example, the proximity-sensor device may be attached to the laminar airflow device. In some embodiments, the proximity-sensor device is an infrared detecting proximity-sensor device, an ultrasonic detecting proximity-sensor device, and/or a hand detecting proximity-sensor device.

In some embodiments, provided herein is a method of protecting the eye-zone or T-zone of a subject in need, comprising the subject wearing the personal protection air-shield device disclosed herein. In some embodiments, provided herein is a method of protecting eyes, nose, or mouth of a subject in need, comprising the subject wearing the personal protection air-shield device disclosed herein. In some embodiments, provided herein is a method of mitigating or preventing infection via the eye-zone or T-zone of a subject in need, comprising the subject wearing the personal protection air-shield device disclosed herein. In some embodiments, provided herein is a method of mitigating or preventing infection of eyes, nose, or mouth of a subject in need, comprising the subject wearing the personal protection air-shield device disclosed herein. In some embodiments, the method provided herein further comprises wearing the head-covering on the head of the subject. In some embodiments, the method provided herein comprises wearing the spectacle device on the head of the subject, such as on the nose, the ears, or both, of the subject.

In some embodiments, provided herein is a method of protecting the eye-zone or T-zone of a subject in need, comprising emitting a laminar airflow that deflects or prevents surrounding external air or air currents from contacting the eye-zone or the T-zone of the subject. In some embodiments, provided herein is a method of protecting eyes, nose, or mouth of a subject in need, comprising emitting a laminar airflow that deflects or prevents surrounding external air or air currents from contacting the eyes, nose, or mouth of the subject. In some embodiments, the method provided herein further comprises generating the laminar airflow. In some embodiments, the method further comprises wearing the head-covering on the head of the subject. In some embodiments, the method further comprises wearing the spectacle device on the head of the subject, such as on the nose, the ears, or both, of the subject.

In some embodiments, the method deflects or prevents surrounding external air or air currents from contacting the eye-zone or the T-zone of the subject wearing the personal protection air-shield device, such as deflects or prevents surrounding external air or air currents from contacting the eyes, nose, or mouth of the subject wearing the personal protection air-shield device. For example, the surrounding external air or air currents comprises an airborne agent, an airborne particle, or an aerosolized droplet. In some embodiments, the method mitigates the risk of being exposed to a breath-laden contaminant adhering to the eye-zone or the T-zone of the subject wearing the personal protection air-shield device, such as mitigates the risk of being exposed to a breath-laden contaminant adhering to the eyes, nose, or mouth of the subject wearing the personal protection air-shield device. In some embodiments, the breath-laden contaminant comprises an airborne agent, an airborne particle, or an aerosolized droplet. In some embodiments, the method mitigates virus exposure to the subject wearing the personal protection air-shield device, for example, the method mitigates the risk of spreading a viral infection from a subject wearing the personal protection air-shield device, or mitigates the risk of contracting a viral infection of a subject wearing the personal protection air-shield device.

In some embodiments, the emitted laminar airflow deflects or prevents surrounding external air or air currents from contacting the eye-zone or the T-zone of the subject wearing the personal protection air-shield device, such as deflects or prevents surrounding external air or air currents from contacting the eyes, nose, or mouth of the subject wearing the personal protection air-shield device. In some embodiments, the emitted laminar airflow mitigates the risk of being exposed to breath-laden contaminants adhering to the eye-zone or the T-zone of the subject wearing the personal protection air-shield device, such as mitigates the risk of being exposed to breath-laden contaminants adhering to the eyes, nose, or mouth of the subject wearing the personal protection air-shield device.

In some embodiments, the emitted laminar airflow mitigates, deflects or blocks exhaled air from a subject wearing the personal protection air-shield device, mitigates, deflects or blocks aerosolized droplets from a subject wearing the personal protection air-shield device, and/or mitigates, deflects or blocks spreading potentially infectious droplets from a subject wearing the personal protection air-shield device. In some embodiments, the emitted laminar airflow mitigates, deflects or blocks exhaled air from another subject, mitigates, deflects or blocks aerosolized droplets from another subject, and/or mitigates, deflects or blocks inhalation of potentially infectious droplets from another subject.

In some embodiments, the emitted laminar airflow mitigates, deflects or blocks an airborne agent, an airborne particle, or an aerosolized droplet. In some embodiments, the surrounding external air or air currents are exterior to the emitted laminar airflow, relative to the subject. For example, the surrounding external air or air currents comprises exhaled air from another subject or comprises aerosolized droplets from another subject. In some embodiments, the airborne agent is or comprises smoke, a noxious agent, or a gas. The airborne particle, for example, is or comprises dust, an insect, an allergen, a microbe, a bacterium and/or a virus. In some embodiments, the aerosolized droplet comprises an airborne agent or an airborne particle, for example, the aerosolized droplet is a potentially infectious aerosolized droplet or is an infectious aerosolized droplet. In some embodiments, the airborne particle is or comprises dust, an insect, an allergen, a microbe, a bacterium, or a virus. In some embodiments, virus is a coronavirus, such as SARS or COVID-19. In some embodiments, the virus is a rhinovirus, such as influenza. In some embodiments, the air-shield device as disclosed herein, and methods of using the same, is suitable for reducing exposure to microbes, and protecting against contamination or infection from microbes. In some embodiments, the microbes include bacteria and/or viruses, such as coronaviruses, for example COVID-19, or rhinoviruses, for example, influenza.

It is understood that subheadings throughout this document do not limit the subject matter discussed to only those sections, but apply, and are contemplated to apply, to each embodiment disclosed in the instant application.

It is understood that modifications which do not substantially affect the activity of the various embodiments of this invention are also provided within the definition of the invention provided herein. Accordingly, the following examples are intended to illustrate but not limit the present invention. All of the references cited to herein are incorporated by reference in their entireties.

### EXAMPLES

### Exemplary Embodiments

One or more than one (including for instance all) of the following embodiments may comprise each of the other embodiments or parts thereof.

A1. In an embodiment, an air-shield device, comprising:
i) an airflow-generating device; and
ii) a laminar airflow device having a vent or a plurality of exit ports, wherein the laminar airflow device is in communication with the airflow-generating device;
wherein the air-shield device is a personal protection air-shield device.

A2. The air-shield device of embodiment A1, wherein the personal protection air-shield device is a personal eye-zone protection air-shield device.

A3. The air-shield device of embodiment A1 or embodiment A2, wherein the personal protection air-shield device is a personal T-zone protection air-shield device.

A4. The air-shield device of any one of embodiments A1-A3, wherein the airflow-generating device comprises a fan.

A5. The air-shield device of any one of embodiments A1-A4, wherein the airflow-generating device comprises a plurality of fans.

A6. The air-shield device of any one of embodiments A1-A5, wherein the airflow-generating device is electric powered.

A7. The air-shield device of any one of embodiments A1-A6, wherein the airflow-generating device is battery powered.

A8. The air-shield device of embodiment A7, wherein the battery is a rechargeable battery.

A9. The air-shield device of any one of embodiments A1-A8, wherein the airflow-generating device is solar powered.

A10. The air-shield device of any one of embodiments A1-A9, wherein the airflow-generating device further comprises an air-intake port and an air-output port.

A11. The air-shield device of any one of embodiments A1-A10, wherein the laminar airflow device is flexible.

A12. The air-shield device of any one of embodiments A1-A11, wherein the laminar airflow device is mouldable.

A13. The air-shield device of any one of embodiments A1-A12, wherein the personal protection air-shield device further comprises an air cleaning device.

A14. The air-shield device of any one of embodiments A1-A13, wherein the airflow-generating device is connected to an air cleaning device via the air-intake port of said airflow-generating device.

A15. The air-shield device of embodiment A14, wherein the air-intake port is configured with an air cleaning device.

A16. The air-shield device of any one of embodiments A13-A15, wherein the air cleaning device is positioned and configured for transferring a flow of cleaned air through the airflow-generating device.

A17. The air-shield device of any one of embodiments A13-A16, wherein the air cleaning device comprises an air cleaning screen.

A18. The air-shield device of any one of embodiments A13-A17, wherein the air cleaning device comprises a light source.

A19. The air-shield device of embodiment A18, wherein the light source comprises light-emitting diodes.

A20. The air-shield device of any one of embodiments A13-A19, wherein the air cleaning device comprises a UVC device.

A21. The air-shield device of embodiment 20, wherein the UVC device comprises light-emitting diodes.

A22. The air-shield device of any one of embodiments A13-A21, wherein the air cleaning device comprises an air cleaning filter.

A23. The air-shield device of embodiment A22, wherein the air cleaning filter is a HEPA filter.

A24. The air-shield device of embodiment A22 or embodiment A23, wherein the air cleaning filter excludes (filters) an airborne agent and/or an airborne particle.

A25. The air-shield device of any one of embodiments A22-A24, wherein the air cleaning filter excludes (filters) an airborne agent.

A26. The air-shield device of any one of embodiments A22-A25, wherein the air cleaning filter excludes (filters) an airborne particle.

A27. The air-shield device of any one of embodiments A22-A26, wherein the air cleaning filter excludes (filters) an airborne particle having a diameter of about 0.01-0.20 um or greater, such as particles having a diameter of about 0.05-0.15 um or greater.

A28. The air-shield device of any one of embodiments A13-A27, wherein the cleaned air flowing from the air cleaning device is exclusive of particles having a diameter of about 0.01-0.20 um or greater, such as particles having a diameter of about 0.05-0.15 um or greater.

A29. The air-shield device of any one of embodiments A13-A28, wherein the cleaned air flowing through the airflow-generating device is exclusive of particles having a diameter of about 0.01-0.20 um or greater, such as particles having a diameter of about 0.05-0.15 um or greater.

A30. The air-shield device of any one of embodiments A13-A29, wherein the cleaned air flowing through the laminar airflow device is exclusive of particles having a diameter of about 0.01-0.20 um or greater, such as particles having a diameter of about 0.05-0.15 um or greater.

A31. The air-shield device of any one of embodiments A13-A30, wherein the laminar airflow device emits cleaned, filtered air from the airflow-generating device and through the vent or the plurality of exit ports.

A32. The air-shield device of any one of embodiments A10-A31, wherein the laminar airflow device is connected to said airflow-generating device via the air-output port.

A33. The air-shield device of any one of embodiments A1-A32, wherein the laminar airflow device is a tube.

A34. The air-shield device of any one of embodiments A1-A33, wherein the laminar airflow device is a tube, wherein at least one end of the tube is connected to and in communication with the airflow-generating device.

A35. The air-shield device of embodiment A33 or embodiment A34, wherein both ends of the tube are connected to and in communication with the airflow-generating device.

A36. The air-shield device of any one of embodiments A33-A35, wherein one end of the tube is connected to and in communication with the airflow-generating device and the second end of the tube is a sealed closed end.

A37. The air-shield device of any one of embodiments A33-A36, wherein the tube is a plastic tube.

A38. The air-shield device of any one of embodiments A33-A37, wherein the tube has an inner diameter of at least 1 mm, 2 mm, 3 mm, 4 mm, 5 mm, 6 mm, 7 mm, 8 mm, 9 mm, 10 mm, or 15 mm.

A39. The air-shield device of any one of embodiments A33-A38, wherein the tube has an inner diameter of less than 10 cm.

A40. The air-shield device of any one of embodiments A33-A39, wherein the tube has an outer diameter of at least 1 mm, 2 mm, 3 mm, 4 mm, 5 mm, 6 mm, 7 mm, 8 mm, 9 mm, 10 mm, or 15 mm.

A41. The air-shield device of any one of embodiments A33-A40, wherein the tube has an outer diameter of less than 10 cm.

A42. The air-shield device of any one of embodiments A1-A41, wherein a portion of the laminar airflow device is exclusive of the vent or the plurality of exit ports.

A43. The air-shield device of any one of embodiments A1-A41, wherein the laminar airflow device emits laminar airflow through the vent or the plurality of exit ports.

A44. The air-shield device of any one of embodiments A1-A43, wherein the vent defines an opening through which laminar air is emitted from the laminar airflow device.

A45. The air-shield device of any one of embodiments A1-A44, wherein each of the plurality of exit ports defines an opening through which laminar air is emitted from the laminar airflow device.

A46. The air-shield device of any one of embodiments A1-A45, wherein one or more exit ports of the plurality of exit ports has a circular perimeter.

A47. The air-shield device of any one of embodiments A1-A46, wherein the opening of one or more exit ports of the plurality of exit ports has a circular perimeter.

A48. The air-shield device of any one of embodiments A1-A47, wherein one or more exit ports of the plurality of exit ports has an oblong perimeter.

A49. The air-shield device of any one of embodiments A1-A48, wherein the opening of one or more exit ports of the plurality of exit ports has an oblong perimeter.

A50. The air-shield device of any one of embodiments A1-A49, wherein one or more exit ports of the plurality of exit ports is a vent.

A51. The air-shield device of any one of embodiments A1-A50, wherein the plurality of exit ports comprises a series of vents.

A52. The air-shield device of any one of embodiments A1-A51, wherein the opening of one or more exit ports of the plurality of exit ports is a slit-shaped vent.

A53. The air-shield device of any one of embodiments A1-A52, wherein the plurality of exit ports comprises at least a first series of exit ports and a second series of exit ports.

A54. The air-shield device of embodiment A53, wherein the at least first series of exit ports and second series of exit ports are positioned to minimize or avoid gaps of emitted laminar airflow.

A55. The air-shield device of any one of embodiments A1-A54, wherein one or more exit ports of the plurality of exit ports has a diameter of at least 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 3 mm, 4 mm, or 5 mm.

A56. The air-shield device of any one of embodiments A1-A55, wherein the vent has a width of at least 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 3 mm, 4 mm, or 5 mm, wherein the width is measured along the perimeter of said tube.

A57. The air-shield device of any one of embodiments A1-A56, wherein the vent has a length of at least 0.2 cm, 0.3 cm, 0.4 cm, 0.5 cm, 1 cm, 2 cm, 3 cm, 4 cm, 5 cm, 6 cm, 7 cm, 8 cm, 9 cm, or 10 cm, wherein the length is measured along the cylindrical length of the tube.

A58. The air-shield device of any one of embodiments A1-A57, wherein the vent defines a slit-shaped opening traversing along the cylindrical length of the tube.

A59. The air-shield device of any one of embodiments A1-A58, wherein the vent is a slit-shaped vent.

A60. The air-shield device of embodiment A59, wherein the slit-shaped vent traverses along the cylindrical length of the tube.

A61. The air-shield device of embodiment A59 or embodiment A60, wherein the slit-shaped vent defines a slit-shaped opening traversing along the cylindrical length of the tube.

A62. The air-shield device of any one of embodiments A1-A61, wherein laminar airflow is emitted through the vent or the plurality of exit ports to create a shield of laminar air about the head of the subject wearing said personal protection air-shield device.

A63. The air-shield device of any one of embodiments A1-A62, wherein laminar airflow is emitted through the vent or the plurality of exit ports to create a shield of laminar air about the face of the subject wearing said personal protection air-shield device.

A64. The air-shield device of any one of embodiments A1-A63, wherein the vent or the plurality of exit ports to direct the current of the emitted laminar airflow at a position about 3 cm relative to the forehead, face, or side of the subject wearing said personal protection air-shield device.

A65. The air-shield device of any one of embodiments A1-A64, wherein the vent or the plurality of exit ports to direct the current of the emitted laminar airflow at a position about 3 cm relative to the forehead, face, side, or back of the head of the subject wearing said personal protection air-shield device.

A66. The air-shield device of any one of embodiments A1-A65, wherein the vent or the plurality of exit ports to direct the current of the emitted laminar airflow at a position at least 3 cm, at least 4 cm, at least 5 cm, at least 6 cm, or at least 7 cm, relative to the forehead, face, or side of the head of the subject wearing said personal protection air-shield device.

A67. The air-shield device of any one of embodiments A1-A66, wherein the vent or the plurality of exit ports to direct the current of the emitted laminar airflow at a position at least 3 cm, at least 4 cm, at least 5 cm, at least 6 cm, or at least 7 cm, relative to the forehead, face, side, or back of the head of the subject wearing said personal protection air-shield device.

A68. The air-shield device of any one of embodiments A1-A67, wherein the emitted laminar airflow is a stream of laminar air or a plurality of streams of laminar air.

A69. The air-shield device of any one of embodiments A1-A68, wherein the emitted laminar airflow is a plane of laminar air or a plurality of parallel planes of laminar air.

A70. The air-shield device of any one of embodiments A1-A69, wherein the emitted laminar airflow is about the face of the head of the subject wearing said personal protection air-shield device.

A71. The air-shield device of any one of embodiments A1-A70, wherein the emitted laminar airflow is: (i) proximate to and about co-planar with the face and sides of the head of the subject wearing said personal protection air-shield device; and/or (ii) proximate to the face and sides of the head and angled towards the chin or based of the head of the subject wearing said personal protection air-shield device.

A72. The air-shield device of any one of embodiments A1-A71, wherein the emitted laminar airflow is about the circumference of the head of the subject wearing said personal protection air-shield device.

A73. The air-shield device of any one of embodiments A1-A72, wherein the emitted laminar airflow spans at least 180 degrees about the circumference of the head of the subject wearing said personal protection air-shield device.

A74. The air-shield device of any one of embodiments A1-A73, wherein the emitted laminar airflow spans at least 180 degrees, 200 degrees, 220 degrees, 240 degrees, 260 degrees, 280 degrees, 300 degrees, 320 degrees, or 340 degrees about the circumference of the head of the subject wearing said personal protection air-shield device.

A75. The air-shield device of any one of embodiments A1-A74, wherein the emitted laminar airflow spans between 180 degrees and 360 degrees about the circumference of the head of the subject wearing said personal protection air-shield device.

A76. The air-shield device of any one of embodiments A1-A75, wherein the emitted laminar airflow spans 360 degrees about the circumference of the head of the subject wearing said personal protection air-shield device.

A77. The air-shield device of any one of embodiments A1-A76, wherein the circumference of the head of the subject wearing said personal protection air-shield device is measured about the cylindrical perimeter comprising both ears and nose of said subject.

A78. The air-shield device of any one of embodiments A1-A77, wherein the laminar airflow is emitted at a rate in the range of about 0.3-0.7 m/s, 0.3-0.5 m/s, 0.3-0.4 m/s, or 0.4-0.5 m/s.

A79. The air-shield device of any one of embodiments A1-A78, wherein the laminar airflow is emitted at a rate of about 0.3 m/s, 0.4 m/s, 0.425 m/s, or 0.5 m/s.

A80. The air-shield device of any one of embodiments A1-A79, wherein the rate of the laminar airflow is an adjustable rate.

A81. The air-shield device of any one of embodiments A1-A80, wherein the laminar air emitted from the air-shield device has a volume in the range of about 0.3-0.7 m³/s, 0.3-0.5 m³/s, or 0.4-0.6 m³/s.

A82. The air-shield device of any one of embodiments A1-A81, wherein the laminar air emitted from the air-shield device has a volume of about 0.3 m³/s, 0.4 m³/s, 0.5 m³/s, 0.6 m³/s, or 0.7 m³/s.

A83. The air-shield device of any one of embodiments A1-A82, wherein the volume of the laminar air emitted from the air-shield device is an adjustable volume.

A84. The air-shield device of any one of embodiments A1-A83, wherein the personal protection air-shield device further comprises a head-covering or a spectacle device.

A85. The air-shield device of any one of embodiments A1-A84, wherein the airflow-generating device is attachable to a head-covering.

A86. The air-shield device of embodiment A84 or embodiment A85, wherein the personal protection air-shield device comprises the head-covering.

A87. The air-shield device of any one of embodiments A84-A86, wherein the airflow-generating device is attached to the head-covering.

A88. The air-shield device of any one of embodiments A84-A87, wherein the airflow-generating device is positioned in the range of between 0.001-1 m from the head-covering.

A89. The air-shield device of any one of embodiments A84-A88, wherein the laminar airflow device is mouldable to adopt the shape of the head-covering.

A90. The air-shield device of any one of embodiments A84-A89, wherein the laminar airflow device is mouldable to adopt the shape of an edge of the head-covering.

A91. The air-shield device of any one of embodiments A84-A90, wherein the laminar airflow device is mouldable to traverse along an edge of the head-covering.

A92. The air-shield device of any one of embodiments A84-A91, wherein the laminar airflow device is mouldable to traverse along the perimeter of the head-covering.

A93. The air-shield device of any one of embodiments A84-A92, wherein the laminar airflow device is mouldable to adopt the shape of the perimeter of the head-covering.

A94. The air-shield device of any one of embodiments A84-A93, wherein at least a portion of the laminar airflow device is positioned about the circumference of the head-covering.

A95. The air-shield device of any one of embodiments A84-A94, wherein the laminar airflow device is attachable to the head-covering.

A96. The air-shield device of any one of embodiments A84-A95, wherein the laminar airflow device is attached to the head-covering.

A97. The air-shield device of any one of embodiments A84-A96, wherein the laminar airflow device is attached or mounted to an edge of the head-covering.

A98. The air-shield device of any one of embodiments A84-A97, wherein the laminar airflow device is attached or mounted to the underside of the head-covering.

A99. The air-shield device of any one of embodiments A84-A98, wherein the laminar airflow device is attached or mounted to the underside of a brim of the head-covering.

A100. The air-shield device of any one of embodiments A84-A99, wherein the laminar airflow device connected to the head covering is positioned to direct the emitted airflow through the vent or the plurality of exit ports at about 3 cm relative to the forehead, face, side, or back of the head of the subject wearing said personal protection air-shield device.

A101. The air-shield device of any one of embodiments A84-A100, wherein the laminar airflow device connected to the head covering is positioned to direct emitted airflow through the vent or the plurality of exit ports at least 3 cm, at least 4 cm, at least 5 cm, at least 6 cm, or at least 7 cm, relative to the forehead, face, side, or back of the head of the subject wearing said personal protection air-shield device.

A102. The air-shield device of any one of embodiments A84-A101, wherein the vent has a length extending at 10%, 20%, 30%, 40%, 50%, 60%, 70%, 80%, 90%, or 100% the circumference of the head-covering.

A103. The air-shield device of any one of embodiments A84-A102, wherein the circumference of the head-covering is measured along an edge of said head-covering.

A104. The air-shield device of any one of embodiments A84-A103, wherein the vent has a length extending at least 10%, 20%, 30%, 40%, 50%, 60%, 70%, 80%, or 90%, of the circumference of the head of the subject wearing said personal protection air-shield device.

A105. The air-shield device of any one of embodiments A84-A104, wherein the vent has a length extending 100% of the circumference of the head of the subject wearing said personal protection air-shield device.

A106. The air-shield device of any one of embodiments A84-A105, wherein the circumference of the head of the subject wearing said personal protection air-shield device is measured about the cylindrical perimeter comprising both ears and nose of said subject.

A107. The air-shield device of any one of embodiments A84-A106, wherein the emitted laminar airflow has a trajectory in the direction from the head towards the feet of the subject wearing said personal protection air-shield device comprising or connected to the head-covering.

A108. The air-shield device of any one of embodiments A84-A107, wherein the emitted laminar airflow is about parallel to the forehead, face, side, or back of the head of the subject wearing said personal protection air-shield device comprising or connected to the head-covering.

A109. The air-shield device of any one of embodiments A84-A108, wherein the emitted laminar airflow is about 1-15 degrees from parallel to the forehead, face, side, or back of the head of the subject wearing said personal protection air-shield device comprising or connected to the head-covering.

A110. The air-shield device of any one of embodiments A84-A109, wherein the emitted laminar airflow is: (i) angled away from the forehead, face, side, or back of the head of the subject wearing said personal protection air-shield device; (ii) proximate to and about co-planar with the forehead, face, sides, or back of the head of the subject wearing said personal protection air-shield device; and/or (iii) proximate to the forehead, upper face, upper sides, or upper back of the head and angled towards the chin or based of the head of the subject wearing said personal protection air-shield device.

A111. The air-shield device of any one of embodiments A84-A110, wherein the emitted laminar airflow is angled about 1-15 degrees from parallel away from the forehead, face, side, or back of the head of the subject wearing said personal protection air-shield device comprising or connected to the head-covering.

A112. The air-shield device of any one of embodiments A84-A111, wherein the laminar airflow emitted from laminar airflow device is at least 3 cm, at least 4 cm, at least 5 cm, at least 6 cm, or at least 7 cm, from the forehead, face, side, or back of the head of the subject wearing said personal protection air-shield device comprising or connected to the head-covering.

A113. The air-shield device of any one of embodiments A84-A112, wherein the head-covering is fixable upon the head of a subject.

A114. The air-shield device of any one of embodiments A84-A113, wherein the head-covering is exclusive of a full head-encapsulating head-covering.

A115. The air-shield device of any one of embodiments A84-A114, wherein the head-covering is exclusive of a hermetically sealed head-covering.

A116. The air-shield device of any one of embodiments A84-A115, wherein the head-covering has a brim.

A117. The air-shield device of any one of embodiments A84-A116, wherein the head-covering is a hat, a helmet, or head garmet.

A118. The air-shield device of embodiment A117, wherein the hat is a brimmed hat.

A119. The air-shield device of embodiment A117, wherein the hat is a baseball hat.

A120. The air-shield device of embodiment A117, wherein the hat is a safari hat.

A121. The air-shield device of any one of embodiments A84-A118, wherein the head-covering directs current of the airflow emitted from the laminar airflow device at least 2 cm, at least 3 cm, at least 4 cm, at least 5 cm, at least 6 cm, or at least 7 cm, relative to the forehead, face, side, or back of the head of the subject wearing said personal protection air-shield device.

A122. The air-shield device of any one of embodiments A1-A84, wherein the airflow-generating device is attachable to a spectacle device.

A123. The air-shield device of any one of embodiments A1-A84 or embodiment A119, wherein the personal protection air-shield device comprises the spectacle device.

A124. The air-shield device of embodiment A84, embodiment A122 or embodiment A123, wherein the airflow-generating device is attached to the spectacle device.

A125. The air-shield device of embodiment A84 or any one of embodiments A122-A124, wherein the airflow-generating device is positioned in the range of between 0.001-1 m from the spectacle device.

A126. The air-shield device of embodiment A84 or any one of embodiments A122-A123, wherein the laminar airflow device is mouldable to adopt the shape of the spectacle frame.

A127. The air-shield device of embodiment A84 or any one of embodiments A122-A126, wherein the laminar airflow device is attachable to the spectacle device.

A128. The air-shield device of embodiment A84 or any one of embodiments A122-A127, wherein the laminar airflow device is attached to the spectacle device.

A129. The air-shield device of embodiment A84 or any one of embodiments A122-A128, wherein the laminar airflow device has a spectacle frame shape.

A130. The air-shield device of embodiment A84 or any one of embodiments A122-A129, wherein the laminar airflow device is a spectacle frame.

A131. The air-shield device of embodiment A84 or any one of embodiments A122-A130, wherein the spectacle device is a spectacle frame housing the laminar airflow device.

A132. The air-shield device of embodiment A84 or any one of embodiments A122-A131, wherein the spectacle device is a spectacle frame.

A133. The air-shield device of embodiment A84 or any one of embodiments A122-A132, wherein the laminar airflow device is a spectacle frame-shaped laminar airflow device.

A134. The air-shield device of embodiment A84 or any one of embodiments A122-A133, wherein the laminar airflow device connected to the spectacle device is positioned to direct the emitted airflow through the vent or the plurality of exit ports at about 3 cm relative to the forehead, face, or side of the subject wearing said personal protection air-shield device.

A135. The air-shield device of embodiment A84 or any one of embodiments A122-A134, wherein the laminar airflow device connected to the spectacle device is positioned to direct emitted airflow through the vent or the plurality of exit ports at least 3 cm, at least 4 cm, at least 5 cm, at least 6 cm, or at least 7 cm, relative to the forehead, face, or side of the subject wearing said personal protection air-shield device.

A136. The air-shield device of embodiment A84 or any one of embodiments A122-A135, wherein the vent has a length extending at least 10%, 20%, 30%, 40%, 50%, 60%, 70%, 80%, or 90%, of the spectacle device.

A137. The air-shield device of embodiment A84 or any one of embodiments A122-A136, wherein the vent has a length extending 100% of the spectacle device.

A138. The air-shield device of embodiment A84 or any one of embodiments A122-A137, wherein the emitted laminar airflow is about parallel to the forehead, face, or side of the subject wearing said personal protection air-shield device comprising or connected to the spectacle device.

A139. The air-shield device of embodiment A84 or any one of embodiments A122-A138, wherein the emitted laminar airflow is about 1-15 degrees from parallel to the forehead, face, or side of the subject wearing said personal protection air-shield device comprising or connected to the spectacle device.

A140. The air-shield device of embodiment A84 or any one of embodiments A122-A139, wherein the emitted laminar airflow is angled away from the forehead, face, or side of the subject wearing said personal protection air-shield device comprising or connected to the spectacle device.

A141. The air-shield device of embodiment A84 or any one of embodiments A122-A140, wherein the emitted laminar airflow is angled about 1-15 degrees from parallel away from the forehead, face, or side of the subject wearing said personal protection air-shield device comprising or connected to the spectacle device.

A142. The air-shield device of embodiment A84 or any one of embodiments A122-A141, wherein the laminar airflow emitted from laminar airflow device is at least 3 cm, at least 4 cm, at least 5 cm, at least 6 cm, or at least 7 cm, from the forehead, face, or side of the subject wearing said personal protection air-shield device comprising or connected to the spectacle device.

A143. The air-shield device of any one of embodiments A1-A142, wherein the personal protection air-shield device further comprises an air-temperature controlling device.

A144. The air-shield device of embodiment A143, wherein the air-temperature controlling device is positioned proximate to the air-intake port.

A145. The air-shield device of embodiment A143 or embodiment A144, wherein the air-temperature controlling device is positioned proximate to the air-output port.

A146. The air-shield device of any one of embodiments A143-A145, wherein the air-temperature controlling device is an air-heating device.

A147. The air-shield device of embodiment A146, wherein the emitted laminar airflow has a higher temperature relative to the temperature of the air entering the air-intake port.

A148. The air-shield device of any one of embodiments A143-A145, wherein the air-temperature controlling device is an air-cooling device.

A149. The air-shield device of embodiment A148, wherein the emitted laminar airflow has a lower temperature relative to the temperature of the air entering the air-intake port.

A150. The air-shield device of any one of embodiments A1-A149, wherein the personal protection air-shield device further comprises an air-humidity controlling device.

A151. The air-shield device of embodiment A150, wherein the air-humidity controlling device is positioned proximate to the air-intake port.

A152. The air-shield device of embodiment A150 or embodiment A148, wherein the air-humidity controlling device is positioned proximate to the air-output port.

A153. The air-shield device of any one of embodiments A150-A152, wherein the emitted laminar airflow has more humidity relative to the humidity of the air entering the air-intake port.

A154. The air-shield device of any one of embodiments A150-A152, wherein the emitted laminar airflow has less humidity relative to the humidity of the air entering the air-intake port.

A155. The air-shield device of any one of embodiments A150-A154, wherein the air-humidity controlling device comprises a fan system.

A156. The air-shield device of any one of embodiments A1-A155, wherein the personal protection air-shield device further comprises an air-ionizing device.

A157. The air-shield device of embodiment A156, wherein the air-ionizing device is positioned proximate to the air-intake port.

A158. The air-shield device of embodiment A156 or embodiment A157, wherein the air-ionizing device is positioned proximate to the air-output port.

A159. The air-shield device of any one of embodiments A156-A158, wherein the air-ionizing device is a carbon-fiber ionizer.

A160. The air-shield device of any one of embodiments A156-A159, wherein the emitted laminar airflow comprises ionized air.

A161. The air-shield device of any one of embodiments A1-A160, wherein the personal protection air-shield device further comprises a proximity-sensor device.

A162. The air-shield device of embodiment A161, wherein the proximity-sensor device is attached to the laminar airflow device.

A163. The air-shield device of embodiment A161 or embodiment A162, wherein the proximity-sensor device is an infrared detecting proximity-sensor device.

A164. The air-shield device of any one of embodiments A162-A163, wherein the proximity-sensor device is an ultrasonic detecting proximity-sensor device.

A165. The air-shield device of any one of embodiments A161-A164, wherein the proximity-sensor device is a hand detecting proximity-sensor device.

A166. A method of protecting the eye-zone or T-zone of a subject in need, comprising the subject wearing the personal protection air-shield device of any one of embodiments A1-A165.

A167. A method of protecting eyes, nose, or mouth of a subject in need, comprising the subject wearing the personal protection air-shield device of any one of embodiments A1-A165.

A168. A method of mitigating or preventing infection via the eye-zone or T-zone of a subject in need, comprising the subject wearing the personal protection air-shield device of any one of embodiments A1-A165.

A169. A method of mitigating or preventing infection of eyes, nose, or mouth of a subject in need, comprising the subject wearing the personal protection air-shield device of any one of embodiments A1-A165.

A170. The method of any one of embodiments A166-A169, wherein the method further comprises wearing the head-covering on the head of the subject.

A171. The method of any one of embodiments A166-A169, wherein the method further comprises wearing the spectacle device on the head of the subject, such as on the nose, the ears, or both, of the subject.

A172. A method of protecting the eye-zone or T-zone of a subject in need, comprising emitting a laminar airflow that deflects or prevents surrounding external air or air currents from contacting the eye-zone or the T-zone of the subject.

A173. A method of protecting eyes, nose, or mouth of a subject in need, comprising emitting a laminar airflow that deflects or prevents surrounding external air or air currents from contacting the eyes, nose, or mouth of the subject.

A174. The method of any one of embodiments A166-A173, wherein the method further comprises generating the laminar airflow.

A175. The method of any one of embodiments A166-A174, wherein the method further comprises wearing the head-covering on the head of the subject.

A176. The method of embodiment A175, wherein the laminar airflow device is attached, connected, or moulded to said head-covering.

A177. The method of any one of embodiments A166-A174, wherein the method further comprises wearing the spectacle device on the head of the subject, such as on the nose, the ears, or both, of the subject.

A178. The method of embodiment A177, wherein the laminar airflow device is attached, connected, or moulded to said spectacle device.

A179. The method of any one of embodiments A166-A178, wherein the method deflects or prevents surrounding external air or air currents from contacting the eye-zone or the T-zone of the subject wearing the personal protection air-shield device.

A180. The method of any one of embodiments A166-A179, wherein the method deflects or prevents surrounding external air or air currents from contacting the eyes, nose, or mouth of the subject wearing the personal protection air-shield device.

A181. The method of any one of embodiments A166-A180, wherein the surrounding external air or air currents comprises an airborne agent, an airborne particle, or an aerosolized droplet.

A182. The method of any one of embodiments A166-A181, wherein the method mitigates the risk of being exposed to a breath-laden contaminant adhering to the eye-zone or the T-zone of the subject wearing the personal protection air-shield device.

A183. The method of any one of embodiments A166-A182, wherein the method mitigates the risk of being exposed to a breath-laden contaminant adhering to the eyes, nose, or mouth of the subject wearing the personal protection air-shield device.

A184. The method of any one of embodiments A166-A183, wherein the breath-laden contaminant comprises an airborne agent, an airborne particle, or an aerosolized droplet.

A185. The method of any one of embodiments A166-A184, wherein the method mitigates virus exposure to the subject wearing the personal protection air-shield device.

A186. The method of any one of embodiments A166-A185, wherein the method mitigates the risk of spreading a viral infection from a subject wearing the personal protection air-shield device.

A187. The method of any one of embodiments A166-A186, wherein the method mitigates the risk of contracting a viral infection of a subject wearing the personal protection air-shield device.

A188. The method of any one of embodiments A166-A187, wherein the emitted laminar airflow deflects or prevents surrounding external air or air currents from contacting the eye-zone or the T-zone of the subject wearing the personal protection air-shield device.

A189. The method of any one of embodiments A166-A188, wherein the emitted laminar airflow deflects or prevents surrounding external air or air currents from contacting the eyes, nose, or mouth of the subject wearing the personal protection air-shield device.

A190. The method of any one of embodiments A166-A189, wherein the emitted laminar airflow mitigates the risk of being exposed to breath-laden contaminants adhering to the eye-zone or the T-zone of the subject wearing the personal protection air-shield device.

A191. The method of any one of embodiments A166-A190, wherein the emitted laminar airflow mitigates the risk of being exposed to breath-laden contaminants adhering to the eyes, nose, or mouth of the subject wearing the personal protection air-shield device.

A192. The method of any one of embodiments A166-A191, wherein the emitted laminar airflow mitigates, deflects or blocks exhaled air from a subject wearing the personal protection air-shield device.

A193. The method of any one of embodiments A166-A192, wherein the emitted laminar airflow mitigates, deflects or blocks aerosolized droplets from a subject wearing the personal protection air-shield device.

A194. The method of any one of embodiments A166-A193, wherein the emitted laminar airflow mitigates, deflects or blocks spreading potentially infectious droplets from a subject wearing the personal protection air-shield device.

A195. The method of any one of embodiments A166-A193, wherein the emitted laminar airflow mitigates, deflects or blocks exhaled air from another subject.

A196. The method of any one of embodiments A166-A195, wherein the emitted laminar airflow mitigates, deflects or blocks aerosolized droplets from another subject.

A197. The method of any one of embodiments A166-A196, wherein the emitted laminar airflow mitigates, deflects or blocks inhalation of potentially infectious droplets from another subject.

A198. The method of any one of embodiments A166-A197, wherein the emitted laminar airflow mitigates, deflects or blocks an airborne agent, an airborne particle, or an aerosolized droplet.

199. The method of any one of embodiments A166-A198, wherein the surrounding external air or air currents are exterior to the emitted laminar airflow, relative to the subject.

A200. The method of any one of embodiments A166-A199, wherein the surrounding external air or air currents comprises exhaled air from another subject.

A201. The method of any one of embodiments A166-A200, wherein the surrounding external air or air currents comprises aerosolized droplets from another subject.

A202. The method of any one of embodiments A166-A201, wherein the airborne agent is or comprises smoke, a noxious agent, or a gas.

A203. The method of any one of embodiments A181-A202, wherein the airborne particle is or comprises dust, an insect, an allergen, a microbe, a bacterium and/or a virus.

A204. The method of any one of embodiments A181-A203, wherein the aerosolized droplet comprises an airborne agent or an airborne particle.

A205. The method of any one of embodiments A181-A204, wherein the aerosolized droplet is a potentially infectious aerosolized droplet.

A206. The method of any one of embodiments A181-A205, wherein the aerosolized droplet is an infectious aerosolized droplet.

A207. The method of any one of embodiments A181-A206, wherein the airborne particle is or comprises dust.

A208. The method of any one of embodiments A181-A207, wherein the airborne particle is or comprises an insect.

A209. The method of any one of embodiments A181-A208, wherein the airborne particle is or comprises an allergen.

A210. The method of any one of embodiments A181-A209, wherein the airborne particle is or comprises a microbe.

A211. The method of any one of embodiments A181-A210, wherein the airborne particle is or comprises a bacterium.

A212. The method of any one of embodiments A181-A211, wherein the airborne particle is or comprises a virus.

A213. The method of embodiment A212, wherein the virus is a coronavirus.

A214. The method of embodiment A213, wherein the coronavirus is SARS.

A215. The method of embodiment A214, wherein the coronavirus is COVID-19.

A216. The method of embodiment A212, wherein the virus is a rhinovirus.

A217. The method of embodiment A216, wherein the rhinovirus is influenza.

### INCORPORATION BY REFERENCE

Various references such as patents, patent applications, and publications are cited herein, the disclosures of which are hereby incorporated by reference herein in their entireties.

I claim:

1. An air-shield device, comprising:
i) an airflow-generating device; and
ii) a laminar airflow device having a vent or a plurality of exit ports, wherein the laminar airflow device is in communication with the airflow-generating device;
wherein the air-shield device is a personal protection air-shield device.

2. The air-shield device of claim 1, wherein the personal protection air-shield device is a personal eye-zone protection air-shield device.

3. The air-shield device of claim 1 or claim 2, wherein the personal protection air-shield device is a personal T-zone protection air-shield device.

4. The air-shield device of any one of claims 1-3, wherein the airflow-generating device comprises a fan.

5. The air-shield device of any one of claims 1-4, wherein the airflow-generating device comprises a plurality of fans.

6. The air-shield device of any one of claims 1-5, wherein the airflow-generating device is electric powered.

7. The air-shield device of any one of claims 1-6, wherein the airflow-generating device is battery powered.

8. The air-shield device of claim 7, wherein the battery is a rechargeable battery.

9. The air-shield device of any one of claims 1-8, wherein the airflow-generating device is solar powered.

10. The air-shield device of any one of claims 1-9, wherein the airflow-generating device further comprises an air-intake port and an air-output port.

11. The air-shield device of any one of claims 1-10, wherein the laminar airflow device is flexible.

12. The air-shield device of any one of claims 1-11, wherein the laminar airflow device is mouldable.

13. The air-shield device of any one of claims 1-12, wherein the personal protection air-shield device further comprises an air cleaning device.

14. The air-shield device of any one of claims 1-13, wherein the airflow-generating device is connected to an air cleaning device via the air-intake port of said airflow-generating device.

15. The air-shield device of claim 14, wherein the air-intake port is configured with an air cleaning device.

16. The air-shield device of any one of claims 13-15, wherein the air cleaning device is positioned and configured for transferring a flow of cleaned air through the airflow-generating device.

17. The air-shield device of any one of claims 13-16, wherein the air cleaning device comprises an air cleaning screen.

18. The air-shield device of any one of claims 13-17, wherein the air cleaning device comprises a light source.

19. The air-shield device of claim 18, wherein the light source comprises light-emitting diodes.

20. The air-shield device of any one of claims 13-19, wherein the air cleaning device comprises a UVC device.

21. The air-shield device of claim 20, wherein the UVC device comprises light-emitting diodes.

22. The air-shield device of any one of claims 13-21, wherein the air cleaning device comprises an air cleaning filter.

23. The air-shield device of claim 22, wherein the air cleaning filter is a HEPA filter.

24. The air-shield device of claim 22 or claim 23, wherein the air cleaning filter excludes (filters) an airborne agent and/or an airborne particle.

25. The air-shield device of any one of claims 22-24, wherein the air cleaning filter excludes (filters) an airborne agent.

26. The air-shield device of any one of claims 22-25, wherein the air cleaning filter excludes (filters) an airborne particle.

27. The air-shield device of any one of claims 22-26, wherein the air cleaning filter excludes (filters) an airborne particle having a diameter of about 0.01-0.20 um or greater, such as particles having a diameter of about 0.05-0.15 um or greater.

28. The air-shield device of any one of claims 13-27, wherein the cleaned air flowing from the air cleaning device is exclusive of particles having a diameter of about 0.01-0.20 um or greater, such as particles having a diameter of about 0.05-0.15 um or greater.

29. The air-shield device of any one of claims 13-28, wherein the cleaned air flowing through the airflow-generating device is exclusive of particles having a diameter of about 0.01-0.20 um or greater, such as particles having a diameter of about 0.05-0.15 um or greater.

30. The air-shield device of any one of claims 13-29, wherein the cleaned air flowing through the laminar airflow device is exclusive of particles having a diameter of about 0.01-0.20 um or greater, such as particles having a diameter of about 0.05-0.15 um or greater.

31. The air-shield device of any one of claims 13-30, wherein the laminar airflow device emits cleaned, filtered air from the airflow-generating device and through the vent or the plurality of exit ports.

32. The air-shield device of any one of claims 10-31, wherein the laminar airflow device is connected to said airflow-generating device via the air-output port.

33. The air-shield device of any one of claims 1-32, wherein the laminar airflow device is a tube.

34. The air-shield device of any one of claims 1-33, wherein the laminar airflow device is a tube, wherein at least one end of the tube is connected to and in communication with the airflow-generating device.

35. The air-shield device of claim 33 or claim 34, wherein both ends of the tube are connected to and in communication with the airflow-generating device.

36. The air-shield device of any one of claims 33-35, wherein one end of the tube is connected to and in communication with the airflow-generating device and the second end of the tube is a sealed closed end.

37. The air-shield device of any one of claims 33-36, wherein the tube is a plastic tube.

38. The air-shield device of any one of claims 33-37, wherein the tube has an inner diameter of at least 1 mm, 2 mm, 3 mm, 4 mm, 5 mm, 6 mm, 7 mm, 8 mm, 9 mm, 10 mm, or 15 mm.

39. The air-shield device of any one of claims 33-38, wherein the tube has an inner diameter of less than 10 cm.

40. The air-shield device of any one of claims 33-39, wherein the tube has an outer diameter of at least 1 mm, 2 mm, 3 mm, 4 mm, 5 mm, 6 mm, 7 mm, 8 mm, 9 mm, 10 mm, or 15 mm.

41. The air-shield device of any one of claims 33-40, wherein the tube has an outer diameter of less than 10 cm.

42. The air-shield device of any one of claims 1-41, wherein a portion of the laminar airflow device is exclusive of the vent or the plurality of exit ports.

43. The air-shield device of any one of claims 1-41, wherein the laminar airflow device emits laminar airflow through the vent or the plurality of exit ports.

44. The air-shield device of any one of claims 1-43, wherein the vent defines an opening through which laminar air is emitted from the laminar airflow device.

45. The air-shield device of any one of claims 1-44, wherein each of the plurality of exit ports defines an opening through which laminar air is emitted from the laminar airflow device.

46. The air-shield device of any one of claims 1-45, wherein one or more exit ports of the plurality of exit ports has a circular perimeter.

47. The air-shield device of any one of claims 1-46, wherein the opening of one or more exit ports of the plurality of exit ports has a circular perimeter.

48. The air-shield device of any one of claims 1-47, wherein one or more exit ports of the plurality of exit ports has an oblong perimeter.

49. The air-shield device of any one of claims 1-48, wherein the opening of one or more exit ports of the plurality of exit ports has an oblong perimeter.

50. The air-shield device of any one of claims 1-49, wherein one or more exit ports of the plurality of exit ports is a vent.

51. The air-shield device of any one of claims 1-50, wherein the plurality of exit ports comprises a series of vents.

52. The air-shield device of any one of claims 1-51, wherein the opening of one or more exit ports of the plurality of exit ports is a slit-shaped vent.

53. The air-shield device of any one of claims 1-52, wherein the plurality of exit ports comprises at least a first series of exit ports and a second series of exit ports.

54. The air-shield device of claim 53, wherein the at least first series of exit ports and second series of exit ports are positioned to minimize or avoid gaps of emitted laminar airflow.

55. The air-shield device of any one of claims 1-54, wherein one or more exit ports of the plurality of exit ports has a diameter of at least 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 3 mm, 4 mm, or 5 mm.

56. The air-shield device of any one of claims 1-55, wherein the vent has a width of at least 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 3 mm, 4 mm, or 5 mm, wherein the width is measured along the perimeter of said tube.

57. The air-shield device of any one of claims 1-56, wherein the vent has a length of at least 0.2 cm, 0.3 cm, 0.4 cm, 0.5 cm, 1 cm, 2 cm, 3 cm, 4 cm, 5 cm, 6 cm, 7 cm, 8 cm, 9 cm, or 10 cm, wherein the length is measured along the cylindrical length of the tube.

58. The air-shield device of any one of claims 1-57, wherein the vent defines a slit-shaped opening traversing along the cylindrical length of the tube.

59. The air-shield device of any one of claims 1-58, wherein the vent is a slit-shaped vent.

60. The air-shield device of claim 59, wherein the slit-shaped vent traverses along the cylindrical length of the tube.

61. The air-shield device of claim 59 or claim 60, wherein the slit-shaped vent defines a slit-shaped opening traversing along the cylindrical length of the tube.

62. The air-shield device of any one of claims 1-61, wherein laminar airflow is emitted through the vent or the plurality of exit ports to create a shield of laminar air about the head of the subject wearing said personal protection air-shield device.

63. The air-shield device of any one of claims 1-62, wherein laminar airflow is emitted through the vent or the plurality of exit ports to create a shield of laminar air about the face of the subject wearing said personal protection air-shield device.

64. The air-shield device of any one of claims 1-63, wherein the vent or the plurality of exit ports to direct the current of the emitted laminar airflow at a position about 3 cm relative to the forehead, face, or side of the subject wearing said personal protection air-shield device.

65. The air-shield device of any one of claims 1-64, wherein the vent or the plurality of exit ports to direct the current of the emitted laminar airflow at a position about 3 cm relative to the forehead, face, side, or back of the head of the subject wearing said personal protection air-shield device.

66. The air-shield device of any one of claims 1-65, wherein the vent or the plurality of exit ports to direct the current of the emitted laminar airflow at a position at least 3 cm, at least 4 cm, at least 5 cm, at least 6 cm, or at least 7 cm, relative to the forehead, face, or side of the head of the subject wearing said personal protection air-shield device.

67. The air-shield device of any one of claims 1-66, wherein the vent or the plurality of exit ports to direct the current of the emitted laminar airflow at a position at least 3 cm, at least 4 cm, at least 5 cm, at least 6 cm, or at least 7 cm, relative to the forehead, face, side, or back of the head of the subject wearing said personal protection air-shield device.

68. The air-shield device of any one of claims 1-67, wherein the emitted laminar airflow is a stream of laminar air or a plurality of streams of laminar air.

69. The air-shield device of any one of claims 1-68, wherein the emitted laminar airflow is a plane of laminar air or a plurality of parallel planes of laminar air.

70. The air-shield device of any one of claims 1-69, wherein the emitted laminar airflow is about the face of the head of the subject wearing said personal protection air-shield device.

71. The air-shield device of any one of claims 1-70, wherein the emitted laminar airflow is:
i) proximate to and about co-planar with the face and sides of the head of the subject wearing said personal protection air-shield device; and/or
ii) proximate to the face and sides of the head and angled towards the chin or based of the head of the subject wearing said personal protection air-shield device.

72. The air-shield device of any one of claims 1-71, wherein the emitted laminar airflow is about the circumference of the head of the subject wearing said personal protection air-shield device.

73. The air-shield device of any one of claims 1-72, wherein the emitted laminar airflow spans at least 180 degrees about the circumference of the head of the subject wearing said personal protection air-shield device.

74. The air-shield device of any one of claims 1-73, wherein the emitted laminar airflow spans at least 180 degrees, 200 degrees, 220 degrees, 240 degrees, 260 degrees, 280 degrees, 300 degrees, 320 degrees, or 340 degrees about the circumference of the head of the subject wearing said personal protection air-shield device.

75. The air-shield device of any one of claims 1-74, wherein the emitted laminar airflow spans between 180 degrees and 360 degrees about the circumference of the head of the subject wearing said personal protection air-shield device.

76. The air-shield device of any one of claims 1-75, wherein the emitted laminar airflow spans 360 degrees about the circumference of the head of the subject wearing said personal protection air-shield device.

77. The air-shield device of any one of claims 1-76, wherein the circumference of the head of the subject wearing said personal protection air-shield device is measured about the cylindrical perimeter comprising both ears and nose of said subject.

78. The air-shield device of any one of claims 1-77, wherein the laminar airflow is emitted at a rate in the range of about 0.3-0.7 m/s, 0.3-0.5 m/s, 0.3-0.4 m/s, or 0.4-0.5 m/s.

79. The air-shield device of any one of claims 1-78, wherein the laminar airflow is emitted at a rate of about 0.3 m/s, 0.4 m/s, 0.425 m/s, or 0.5 m/s.

80. The air-shield device of any one of claims 1-79, wherein the rate of the laminar airflow is an adjustable rate.

81. The air-shield device of any one of claims 1-80, wherein the laminar air emitted from the air-shield device has a volume in the range of about 0.3-0.7 m³/s, 0.3-0.5 m³/s, or 0.4-0.6 m³/s.

82. The air-shield device of any one of claims 1-81, wherein the laminar air emitted from the air-shield device has a volume of about 0.3 m³/s, 0.4 m³/s, 0.5 m³/s, 0.6 m³/s, or 0.7 m³/s.

83. The air-shield device of any one of claims 1-82, wherein the volume of the laminar air emitted from the air-shield device is an adjustable volume.

84. The air-shield device of any one of claims 1-83, wherein the personal protection air-shield device further comprises a head-covering or a spectacle device.

85. The air-shield device of any one of claims 1-84, wherein the airflow-generating device is attachable to a head-covering.

86. The air-shield device of claim 84 or claim 85, wherein the personal protection air-shield device comprises the head-covering.

87. The air-shield device of any one of claims 84-86, wherein the airflow-generating device is attached to the head-covering.

88. The air-shield device of any one of claims 84-87, wherein the airflow-generating device is positioned in the range of between 0.001-1 m from the head-covering.

89. The air-shield device of any one of claims 84-88, wherein the laminar airflow device is mouldable to adopt the shape of the head-covering.

90. The air-shield device of any one of claims 84-89, wherein the laminar airflow device is mouldable to adopt the shape of an edge of the head-covering.

91. The air-shield device of any one of claims 84-90, wherein the laminar airflow device is mouldable to traverse along an edge of the head-covering.

92. The air-shield device of any one of claims 84-91, wherein the laminar airflow device is mouldable to traverse along the perimeter of the head-covering.

93. The air-shield device of any one of claims 84-92, wherein the laminar airflow device is mouldable to adopt the shape of the perimeter of the head-covering.

94. The air-shield device of any one of claims 84-93, wherein at least a portion of the laminar airflow device is positioned about the circumference of the head-covering.

95. The air-shield device of any one of claims 84-94, wherein the laminar airflow device is attachable to the head-covering.

96. The air-shield device of any one of claims 84-95, wherein the laminar airflow device is attached to the head-covering.

97. The air-shield device of any one of claims 84-96, wherein the laminar airflow device is attached or mounted to an edge of the head-covering.

98. The air-shield device of any one of claims 84-97, wherein the laminar airflow device is attached or mounted to the underside of the head-covering.

99. The air-shield device of any one of claims 84-98, wherein the laminar airflow device is attached or mounted to the underside of a brim of the head-covering.

100. The air-shield device of any one of claims 84-99, wherein the laminar airflow device connected to the head covering is positioned to direct the emitted airflow through the vent or the plurality of exit ports at about 3 cm relative to the forehead, face, side, or back of the head of the subject wearing said personal protection air-shield device.

101. The air-shield device of any one of claims 84-100, wherein the laminar airflow device connected to the head covering is positioned to direct emitted airflow through the vent or the plurality of exit ports at least 3 cm, at least 4 cm, at least 5 cm, at least 6 cm, or at least 7 cm, relative to the forehead, face, side, or back of the head of the subject wearing said personal protection air-shield device.

102. The air-shield device of any one of claims 84-101, wherein the vent has a length extending at 10%, 20%, 30%, 40%, 50%, 60%, 70%, 80%, 90%, or 100% the circumference of the head-covering.

103. The air-shield device of any one of claims 84-102, wherein the circumference of the head-covering is measured along an edge of said head-covering.

104. The air-shield device of any one of claims 84-103, wherein the vent has a length extending at least 10%, 20%, 30%, 40%, 50%, 60%, 70%, 80%, or 90%, of the circumference of the head of the subject wearing said personal protection air-shield device.

105. The air-shield device of any one of claims 84-104, wherein the vent has a length extending 100% of the circumference of the head of the subject wearing said personal protection air-shield device.

106. The air-shield device of any one of claims 84-105, wherein the circumference of the head of the subject wearing said personal protection air-shield device is measured about the cylindrical perimeter comprising both ears and nose of said subject.

107. The air-shield device of any one of claims 84-106, wherein the emitted laminar airflow has a trajectory in the direction from the head towards the feet of the subject wearing said personal protection air-shield device comprising or connected to the head-covering.

108. The air-shield device of any one of claims 84-107, wherein the emitted laminar airflow is about parallel to the forehead, face, side, or back of the head of the subject wearing said personal protection air-shield device comprising or connected to the head-covering.

109. The air-shield device of any one of claims 84-108, wherein the emitted laminar airflow is about 1-15 degrees from parallel to the forehead, face, side, or back of the head of the subject wearing said personal protection air-shield device comprising or connected to the head-covering.

110. The air-shield device of any one of claims 84-109, wherein the emitted laminar airflow is:
i) angled away from the forehead, face, side, or back of the head of the subject wearing said personal protection air-shield device;
ii) proximate to and about co-planar with the forehead, face, sides, or back of the head of the subject wearing said personal protection air-shield device; and/or
iii) proximate to the forehead, upper face, upper sides, or upper back of the head and angled towards the chin or based of the head of the subject wearing said personal protection air-shield device.

111. The air-shield device of any one of claims 84-110, wherein the emitted laminar airflow is angled about 1-15 degrees from parallel away from the forehead, face, side, or back of the head of the subject wearing said personal protection air-shield device comprising or connected to the head-covering.

112. The air-shield device of any one of claims 84-111, wherein the laminar airflow emitted from laminar airflow device is at least 3 cm, at least 4 cm, at least 5 cm, at least 6 cm, or at least 7 cm, from the forehead, face, side, or back of the head of the subject wearing said personal protection air-shield device comprising or connected to the head-covering.

113. The air-shield device of any one of claims 84-112, wherein the head-covering is fixable upon the head of a subject.

114. The air-shield device of any one of claims 84-113, wherein the head-covering is exclusive of a full head-encapsulating head-covering.

115. The air-shield device of any one of claims 84-114, wherein the head-covering is exclusive of a hermetically sealed head-covering.

116. The air-shield device of any one of claims 84-115, wherein the head-covering has a brim.

117. The air-shield device of any one of claims 84-116, wherein the head-covering is a hat, a helmet, or head garment.

118. The air-shield device of claim 117, wherein the hat is a brimmed hat.

119. The air-shield device of claim 117, wherein the hat is a baseball hat.

120. The air-shield device of claim 117, wherein the hat is a safari hat.

121. The air-shield device of any one of claims 84-120, wherein the head-covering directs current of the airflow emitted from the laminar airflow device at least 2 cm, at least 3 cm, at least 4 cm, at least 5 cm, at least 6 cm, or at least 7 cm, relative to the forehead, face, side, or back of the head of the subject wearing said personal protection air-shield device.

122. The air-shield device of any one of claims 1-84, wherein the airflow-generating device is attachable to a spectacle device.

123. The air-shield device of any one of claims 1-84 or claim 122, wherein the personal protection air-shield device comprises the spectacle device.

124. The air-shield device of claim 84, claim 122 or claim 123, wherein the airflow-generating device is attached to the spectacle device.

125. The air-shield device of claim 84 or any one of claims 122-124, wherein the airflow-generating device is positioned in the range of between 0.001-1 m from the spectacle device.

126. The air-shield device of claim 84 or any one of claims 122-125, wherein the laminar airflow device is mouldable to adopt the shape of the spectacle frame.

127. The air-shield device of claim 84 or any one of claims 122-126, wherein the laminar airflow device is attachable to the spectacle device.

128. The air-shield device of claim 84 or any one of claims 122-127, wherein the laminar airflow device is attached to the spectacle device.

129. The air-shield device of claim 84 or any one of claims 122-128, wherein the laminar airflow device has a spectacle frame shape.

130. The air-shield device of claim 84 or any one of claims 122-129, wherein the laminar airflow device is a spectacle frame.

131. The air-shield device of claim 84 or any one of claims 122-130, wherein the spectacle device is a spectacle frame housing the laminar airflow device.

132. The air-shield device of claim 84 or any one of claims 122-131, wherein the spectacle device is a spectacle frame.

133. The air-shield device of claim 84 or any one of claims 122-132, wherein the laminar airflow device is a spectacle frame-shaped laminar airflow device.

134. The air-shield device of claim 84 or any one of claims 122-133, wherein the laminar airflow device connected to the spectacle device is positioned to direct the emitted airflow through the vent or the plurality of exit ports at about 3 cm relative to the forehead, face, or side of the subject wearing said personal protection air-shield device.

135. The air-shield device of claim 84 or any one of claims 122-134, wherein the laminar airflow device connected to the spectacle device is positioned to direct emitted airflow through the vent or the plurality of exit ports at least 3 cm, at least 4 cm, at least 5 cm, at least 6 cm, or at least 7 cm, relative to the forehead, face, or side of the subject wearing said personal protection air-shield device.

136. The air-shield device of claim 84 or any one of claims 122-135, wherein the vent has a length extending at least 10%, 20%, 30%, 40%, 50%, 60%, 70%, 80%, or 90%, of the spectacle device.

137. The air-shield device of claim 84 or any one of claims 122-136, wherein the vent has a length extending 100% of the spectacle device.

138. The air-shield device of claim 84 or any one of claims 122-137, wherein the emitted laminar airflow is about parallel to the forehead, face, or side of the subject wearing said personal protection air-shield device comprising or connected to the spectacle device.

139. The air-shield device of claim 84 or any one of claims 122-138, wherein the emitted laminar airflow is about 1-15 degrees from parallel to the forehead, face, or side of the subject wearing said personal protection air-shield device comprising or connected to the spectacle device.

140. The air-shield device of claim 84 or any one of claims 122-139, wherein the emitted laminar airflow is angled away from the forehead, face, or side of the subject wearing said personal protection air-shield device comprising or connected to the spectacle device.

141. The air-shield device of claim 84 or any one of claims 122-140, wherein the emitted laminar airflow is angled about 1-15 degrees from parallel away from the forehead, face, or side of the subject wearing said personal protection air-shield device comprising or connected to the spectacle device.

142. The air-shield device of claim 84 or any one of claims 122-141, wherein the laminar airflow emitted from laminar airflow device is at least 3 cm, at least 4 cm, at least 5 cm, at least 6 cm, or at least 7 cm, from the forehead, face, or side of the subject wearing said personal protection air-shield device comprising or connected to the spectacle device.

143. The air-shield device of any one of claims 1-142, wherein the personal protection air-shield device further comprises an air-temperature controlling device.

144. The air-shield device of claim 143, wherein the air-temperature controlling device is positioned proximate to the air-intake port.

145. The air-shield device of claim 143 or claim 144, wherein the air-temperature controlling device is positioned proximate to the air-output port.

146. The air-shield device of any one of claims 143-145, wherein the air-temperature controlling device is an air-heating device.

147. The air-shield device of claim 146, wherein the emitted laminar airflow has a higher temperature relative to the temperature of the air entering the air-intake port.

148. The air-shield device of any one of claims 143-145, wherein the air-temperature controlling device is an air-cooling device.

149. The air-shield device of claim 148, wherein the emitted laminar airflow has a lower temperature relative to the temperature of the air entering the air-intake port.

150. The air-shield device of any one of claims 1-149, wherein the personal protection air-shield device further comprises an air-humidity controlling device.

151. The air-shield device of claim 150, wherein the air-humidity controlling device is positioned proximate to the air-intake port.

152. The air-shield device of claim 150 or claim 151, wherein the air-humidity controlling device is positioned proximate to the air-output port.

153. The air-shield device of any one of claims 150-152, wherein the emitted laminar airflow has more humidity relative to the humidity of the air entering the air-intake port.

154. The air-shield device of any one of claims 150-152, wherein the emitted laminar airflow has less humidity relative to the humidity of the air entering the air-intake port.

155. The air-shield device of any one of claims 150-154, wherein the air-humidity controlling device comprises a fan system.

156. The air-shield device of any one of claims 1-155, wherein the personal protection air-shield device further comprises an air-ionizing device.

157. The air-shield device of claim 156, wherein the air-ionizing device is positioned proximate to the air-intake port.

158. The air-shield device of claim 156 or claim 157, wherein the air-ionizing device is positioned proximate to the air-output port.

159. The air-shield device of any one of claims 156-158, wherein the air-ionizing device is a carbon-fiber ionizer.

160. The air-shield device of any one of claims 156-159, wherein the emitted laminar airflow comprises ionized air.

161. The air-shield device of any one of claims 1-160, wherein the personal protection air-shield device further comprises a proximity-sensor device.

162. The air-shield device of claim 161, wherein the proximity-sensor device is attached to the laminar airflow device.

163. The air-shield device of claim 161 or claim 162, wherein the proximity-sensor device is an infrared detecting proximity-sensor device.

164. The air-shield device of any one of claims 161-163, wherein the proximity-sensor device is an ultrasonic detecting proximity-sensor device.

165. The air-shield device of any one of claims 161-164, wherein the proximity-sensor device is a hand detecting proximity-sensor device.

166. A method of protecting the eye-zone or T-zone of a subject in need, comprising the subject wearing the personal protection air-shield device of any one of claims 1-165.

167. A method of protecting eyes, nose, or mouth of a subject in need, comprising the subject wearing the personal protection air-shield device of any one of claims 1-165.

168. A method of mitigating or preventing infection via the eye-zone or T-zone of a subject in need, comprising the subject wearing the personal protection air-shield device of any one of claims 1-165.

169. A method of mitigating or preventing infection of eyes, nose, or mouth of a subject in need, comprising the subject wearing the personal protection air-shield device of any one of claims 1-165.

170. The method of any one of claims 166-169, wherein the method further comprises wearing the head-covering on the head of the subject.

171. The method of any one of claims 166-169, wherein the method further comprises wearing the spectacle device on the head of the subject, such as on the nose, the ears, or both, of the subject.

172. A method of protecting the eye-zone or T-zone of a subject in need, comprising emitting a laminar airflow that deflects or prevents surrounding external air or air currents from contacting the eye-zone or the T-zone of the subject.

173. A method of protecting eyes, nose, or mouth of a subject in need, comprising emitting a laminar airflow that deflects or prevents surrounding external air or air currents from contacting the eyes, nose, or mouth of the subject.

174. The method of any one of claims 166-173, wherein the method further comprises generating the laminar airflow.

175. The method of any one of claims 166-174, wherein the method further comprises wearing the head-covering on the head of the subject.

176. The method of claim 175, wherein the laminar airflow device is attached, connected, or moulded to said head-covering.

177. The method of any one of claims 166-174, wherein the method further comprises wearing the spectacle device on the head of the subject, such as on the nose, the ears, or both, of the subject.

178. The method of claim 177, wherein the laminar airflow device is attached, connected, or moulded to said spectacle device.

179. The method of any one of claims 166-178, wherein the method deflects or prevents surrounding external air or air currents from contacting the eye-zone or the T-zone of the subject wearing the personal protection air-shield device.

180. The method of any one of claims 166-179, wherein the method deflects or prevents surrounding external air or air currents from contacting the eyes, nose, or mouth of the subject wearing the personal protection air-shield device.

181. The method of any one of claims 166-180, wherein the surrounding external air or air currents comprises an airborne agent, an airborne particle, or an aerosolized droplet.

182. The method of any one of claims 166-181, wherein the method mitigates the risk of being exposed to a breath-laden contaminant adhering to the eye-zone or the T-zone of the subject wearing the personal protection air-shield device.

183. The method of any one of claims 166-182, wherein the method mitigates the risk of being exposed to a breath-laden contaminant adhering to the eyes, nose, or mouth of the subject wearing the personal protection air-shield device.

184. The method of any one of claims 166-183, wherein the breath-laden contaminant comprises an airborne agent, an airborne particle, or an aerosolized droplet.

185. The method of any one of claims 166-184, wherein the method mitigates virus exposure to the subject wearing the personal protection air-shield device.

186. The method of any one of claims 166-185, wherein the method mitigates the risk of spreading a viral infection from a subject wearing the personal protection air-shield device.

187. The method of any one of claims 166-186, wherein the method mitigates the risk of contracting a viral infection of a subject wearing the personal protection air-shield device.

188. The method of any one of claims 166-187, wherein the emitted laminar airflow deflects or prevents surrounding external air or air currents from contacting the eye-zone or the T-zone of the subject wearing the personal protection air-shield device.

189. The method of any one of claims 166-188, wherein the emitted laminar airflow deflects or prevents surrounding external air or air currents from contacting the eyes, nose, or mouth of the subject wearing the personal protection air-shield device.

190. The method of any one of claims 166-189, wherein the emitted laminar airflow mitigates the risk of being exposed to breath-laden contaminants adhering to the eye-zone or the T-zone of the subject wearing the personal protection air-shield device.

191. The method of any one of claims 166-190, wherein the emitted laminar airflow mitigates the risk of being exposed to breath-laden contaminants adhering to the eyes, nose, or mouth of the subject wearing the personal protection air-shield device.

192. The method of any one of claims 166-191, wherein the emitted laminar airflow mitigates, deflects or blocks exhaled air from a subject wearing the personal protection air-shield device.

193. The method of any one of claims 166-192, wherein the emitted laminar airflow mitigates, deflects or blocks aerosolized droplets from a subject wearing the personal protection air-shield device.

194. The method of any one of claims 166-193, wherein the emitted laminar airflow mitigates, deflects or blocks spreading potentially infectious droplets from a subject wearing the personal protection air-shield device.

195. The method of any one of claims 166-194, wherein the emitted laminar airflow mitigates, deflects or blocks exhaled air from another subject.

196. The method of any one of claims 166-195, wherein the emitted laminar airflow mitigates, deflects or blocks aerosolized droplets from another subject.

197. The method of any one of claims 166-196, wherein the emitted laminar airflow mitigates, deflects or blocks inhalation of potentially infectious droplets from another subject.

198. The method of any one of claims 166-197, wherein the emitted laminar airflow mitigates, deflects or blocks an airborne agent, an airborne particle, or an aerosolized droplet.

199. The method of any one of claims 166-198, wherein the surrounding external air or air currents are exterior to the emitted laminar airflow, relative to the subject.

200. The method of any one of claims 166-199, wherein the surrounding external air or air currents comprises exhaled air from another subject.

201. The method of any one of claims 166-200, wherein the surrounding external air or air currents comprises aerosolized droplets from another subject.

202. The method of any one of claims 166-201, wherein the airborne agent is or comprises smoke, a noxious agent, or a gas.

203. The method of any one of claims 181-202, wherein the airborne particle is or comprises dust, an insect, an allergen, a microbe, a bacterium and/or a virus.

204. The method of any one of claims 181-203, wherein the aerosolized droplet comprises an airborne agent or an airborne particle.

205. The method of any one of claims 181-204, wherein the aerosolized droplet is a potentially infectious aerosolized droplet.

206. The method of any one of claims 181-205, wherein the aerosolized droplet is an infectious aerosolized droplet.

207. The method of any one of claims 181-206, wherein the airborne particle is or comprises dust.

208. The method of any one of claims 181-207, wherein the airborne particle is or comprises an insect.

209. The method of any one of claims 181-208, wherein the airborne particle is or comprises an allergen.

210. The method of any one of claims 181-209, wherein the airborne particle is or comprises a microbe.

211. The method of any one of claims 181-210, wherein the airborne particle is or comprises a bacterium.

212. The method of any one of claims 181-211, wherein the airborne particle is or comprises a virus.

213. The method of claim 212, wherein the virus is a coronavirus.

214. The method of claim 213, wherein the coronavirus is SARS.

215. The method of claim 214, wherein the coronavirus is COVID-19.

216. The method of claim 212, wherein the virus is a rhinovirus.

217. The method of claim 216, wherein the rhinovirus is influenza.
